(19) United States
(12) Patent Application Publication
(10) Pub. No.: US 2005/0056290 A1
(43) Pub. Date: Mar. 17, 2005

(54) Method of neuromodulation therapy

(76) Inventor: Leslie S. Feinberg, Hermiston, OR (US)

Correspondence Address: Carl D. Crowell, P.O. Box 923, Salem, OR, 97308, US

(21) Appl. No.: 10/662,960

(22) Filed: Sep. 15, 2003

(51) Int. Cl. A61B 19/00

(52) U.S. Cl. 128/898

(57) ABSTRACT

A method of energetic medicine wherein a practitioner corrects faults in various functional categories of the system that controls vegetative functions in the body of the subject. The method is comprised of the steps of applying specific clinical algorithms consisting of queries and corrective statements to assess and then train this system toward optimal function. The medium by which this is achieved is verbal and/or other than conscious communications from the practitioner to subject, as well as application of physical stimuli. Communication from subject to the practitioner is determined by monitoring an indicator of autonomic function.

### BACKGROUND OF THE INVENTION

1. Field of the Invention

The present invention discloses and teaches an improved method of energetic medicine, or neuromodulation therapy (NMT). Energetic medicine recognizes that humans have an internal control system that has the capacity to respond to the circumstances in which they find themselves in such a way as to maintain a homeostatic internal environment. It holds that when we become ill there is always some confusion of that system that compromises this control system's optimal internal management of the body. To be effective energetic medicine generally must first access that control system and establish some sort of interface with it. Second, assess the way in which that control system has deviated from optimal function. Lastly, afford some method of pushing the control system to modulate it back in the direction of optimal performance.

2. Background of the Field

Various systems of energetic medicine date back thousands of years and include is the development of acupuncture in China and Ayurvedic medicine in India. Much attention was focused on development of energetic systems of healing in early cultures due to limitations of economics and technology. In western culture, the marvel of modern medical technology is rivaled only by the fantastic rise in the cost of that medical system to our society. Some aspects of modern medicine will never be replaced by energetic medicine. For example, the victim of a gunshot or an auto accident would do well to proceed directly to the local emergency room, since energetic suturing is not known to be very effective. When the patient turns to those very serious ongoing challenges to health that come from chronic degenerative disease, however, modern medicine very little to offer. Patients with conditions like arthritis, allergies, and auto-immune conditions of the gastrointestinal system, nervous system, connective tissue system, may look to modern medicine only with respect to temporary suppression of symptoms, and then often at the cost of unintended side effects of drugs and surgery.

Energetic Medicine works to gain access to that subconscious autonomic regulatory system, to thoroughly assess its status, and to modulate its function until the patient's health has been tuned to correct any improper expression of the energetic and neurological control systems of the body.

Healing may be the result of the ministrations of a traditional African healer, an Inuit shaman, a Christian Scientist, any one of the methods of energetic healing that have developed in recent decades, a prayer circle at the local church, the present disclosed technique, or even, perhaps, acupuncture and similar procedures. Healing comes from within the body and is an expression of the programming inherent in all living things. The differences in efficacy of any of these methods of energetic medicine relate to such variables as how well the method establishes an interface between the healer and the patient (access), how well the method affords a way to look into that system of programming and visualize errors contributing to the patient's condition (assess), and finally how well structured, clear, and appropriate is the corrective information that is input to the patient (modulate).

Some methods of energetic medicine are clear about the role of projection of the intent of the practitioner in providing corrective information to the control systems of the patient. Other methods foster the illusion that particular elements of those procedures that do not have inherent informational content are significant in their own right, the special gestures, points, and paraphernalia of energetic medicine. A more informed view is that such procedural elements function as metaphor to assist the practitioner in forming intent with corrective content. Awareness of the practitioner that such is the case may, or may not be a characteristic of any particular system of healing.

An essential question for the practitioner of energetic medicine to answer at the very beginning of the process to trigger healing for a patient is, “What is healing, what is the nature of it, and where does it come from?” Life begins with the combination of 23 pairs of chromosomes from each parent resulting in the genome of an embryonic new life consisting of 46 chromosomes occupying the nucleus of a single cell. There is, according to principles of the theory of formative causation, the concurrent presence of energetic fields that model the morphology of the material representation of our bodies. From this beginning unfolds all material structure, all rules of the physiology of growth, repair, disease, degeneration, and aging. The genome of each individual comprises a database utilizing a system of four base pairs ordered along two ribbons of chained deoxyribose sugar and phosphoric acid molecules. This system is otherwise a recording of an information state for a biological computer just as the arrangement of zeros and ones on the hard drive, processors, and memory chips of a computer records an information state for that system. The combination of this chemical “hardware” and energetic “software” comprise the elements of the information storage system from which human life develops.

The physical body, as it develops and during its mature life, operates according to data processing rules implicit in the inherited genetic chemical and morphogenic field characteristics of the individual. The nervous system is a primary agent by which this is manifested. Some functions that occur on a chemical level are simply inherent to the chemical structure of proteins or other compounds that make up the chemical structures such as with the behavior of RNA.

There are limitations that exist, and which define the range of possibilities of function for the organism. Within this range, optimal health can be expected as long as there are no factors that compromise the expression of data contained in the DNA as represented by the structure and function of control tissues such as the nervous system. Within this range of possibilities of normal function, there may be factors that interrupt the normal expression of control data. The result of such interruption is illness of one degree or another. These possibilities include the influences of infectious agents, allergens, data processing failures within the sensory/motor control system, corruption of data storage within the central nervous system, mutation induced corruption within the data storage chemical such as RNA or DNA, and the introduction of inhibitory, or excitatory exogenous analogs of endocrine hormones. Any of these factors are capable of compromising the otherwise proper execution of physiological controls in the body resulting in disease. There are other factors which are obviously capable of compromising the health of the individual but these particular factors which compromise the expression of normal controls in the body are responsible for an enormous percentage of human illness. These are the factors that are within the realm of the disclosed therapy.

History of the Field

There are many forms of energetic medicine for which it is extremely difficult to show any reasonable degree of proof. The chief reason for this is that in many of these methods the effect of treatment is produced gradually over time. This being the case, it is very difficult to isolate what specific feature of that energetic healing method, or other intervening experience in the patient's life is responsible for any observed change in health.

It would be of value to consider the history over the last century or two of man's attempts to find the key to restoring normal autonomic regulation. The nineteenth century witnessed the development of homeopathy by Dr. Samuel Hanneman, the development of osteopathy by Dr. Andrew Still, and the development of chiropractic by Dr. D. D. Palmer. It was the goal of homeopathy to imprint samples of water with the energetic signature of a substance that when introduced to the body produced the same symptoms as those that the patient has presented with. It is the theory of homeopathy that when this preparation has been so diluted that there is in all mathematical probability not a single molecule of the substance whose energetic signature was imprinted upon it. Furthermore, it is the position of homeopathy that the ingestion of this very dilute preparation will trigger the body to stop the illness behavior that resulted in the symptom for which the homeopathic remedy was intended.

It was the position of Dr. Still that illness was the result of diminished circulation caused by dysrelationship of various osseous articulations. His solution was to perform manipulations of the joints and soft tissues. Eventually, osteopathy suffered from a failure to progressively develop its therapeutic philosophy. For a time, osteopathy lost the vision of its founder, and despaired over ever finding the key to restoring normal autonomic function. It became blended with conventional allopathic medicine to the point that they have been indistinguishable. In fairness to the profession, osteopathic pioneers like John Upledger, D.O. and others have broken new ground in alternative medicine with such developments as muscle energy work, and Craniosacral Technique. Such innovations once again distinguish the contributions of osteopathy.

Dr. D. D. Palmer is said to have begun the science of chiropractic with the observation that a displaced thoracic vertebra seemed to have a correlation with loss of hearing in a man named Harvey Lilliard. Palmer went on to develop the position that the chief regulatory system of the body was the nervous system and that this system was charged with the responsibility of expressing what he referred to as “innate intelligence” that designed the body and drove its processes. This was over 100 years ago. There is no science that stands by the tenets it adhered to over a century ago and chiropractic is no exception. Progress in natural science in the past century has described much that was unknown in Palmer's time. Though the explanation must now be different, the conclusion that health proceeds through the expression of a plan, or information-processing program, via the nervous system continues to be entirely supportable today.

It was Dr. Palmer's contention that the expression of regulatory forces throughout the body was mediated by the nervous system and was patient to compromise at the intravertebral foramen due to disturbance in the physiology of spinal articulations. Palmer referred to the compromise in expression of regulation when it occurred at the level of the intravertebral foramen as “quantity interference”. Palmer also described another type of compromise in the expression of regulation of the body that did not occur at this spinal level. He referred to this as “quality interference” and said that this interference occurred within the central nervous system prior to the branching into peripheral nerves. This, in part, is the territory staked out by the disclosed method of neuromodulation therapy—the correction of these subluxations of “quality interference” to the unadulterated expression of the human nervous system.

Over the many years that have passed since chiropractic began, many different approaches have been developed by chiropractors to correct the dysregulation of the nervous system. A tremendous richness of therapeutic approaches has unfolded, developed by doctors in the field driven by the yearning to find that perfect key to restoring the full, complete, and accurate expression of our blueprint of life.

The second half of the first century of chiropractic saw the introduction of techniques that were not primarily mechanical in nature. It was recognized that the process of performing vertebral manipulation involved applying physical forces to the body that activated various neurological sensors and by reflex, caused a change in the motor output of the nervous system thus correcting functional disturbance. This often resulted in improvement in physiology, particularly the physiology of posture, and motion of the spine. Many times it resulted in improvement of visceral function. Many of the techniques developed in the first half of the first century of chiropractic took an interest in the creation of efficient ways of producing osseous manipulation. Many of the techniques developed in the second half of the first century of chiropractic took an interest in developing systems that were more purely neurologically reflexive in nature, or based on the principals of energetic medicine.

The energetic techniques in chiropractic draw upon two of the earlier methods of energetic treatment, acupuncture and homeopathy. These methods, when filtered through the grid of chiropractic perspective, resulted in what may be termed “point based” techniques, “vial based” techniques, and techniques that utilized both approaches. One of the earliest of “point based” techniques that involved the use of particular points on the body wall that were thought to be correlated to visceral function was known as the Bennett reflexes. Various other systems of “point based” treatment protocols developed within the chiropractic profession including systems of treatment such as Touch for Health, the Versendaal method, and a number of types of reflexology. The Versendaal method utilizes a vast system of points representing various tissues, organs, nutrients, and processes. Other methods such as Clinical Kinesiology from Allen Beardall, D.C. utilize enormous numbers of hand positions known as mudras, or hand modes.

The inherent weakness of the “Pont Based” techniques is that they require predetermination of the meaning of each point that is chosen as a metaphorical representation of some body process or structure. This severely limits corrective communications with the ACS of the patient as compared to the semantic structure of neuromodulation technique.

The inventor asserts that there is nothing inherent in any of these hand positions, or body points that makes them of any therapeutic value. The concepts of body points, mudras, and gestures have meaning only to the extent that these things represent metaphorical mental placeholders for the doctor to establish intent.

There is some danger in forgetting that this is the case and simply assuming that such metaphors are literally real. Such metaphors are constituted from the presumptions of the people who developed the techniques that utilize these concepts and are not inherent in the anatomy of human beings. These presumptions are transferred to the practitioners who use the method in their training, often just that way—as presumptions that are not even acknowledged, and are generally assimilated without question, and used clinically without awareness.

George Goodheart, D.C., a second-generation chiropractor from Michigan, was the subject of an Apr. 16, 2001 Time magazine article featuring Time's 100 great innovators in medicine. Forty years previously, in 1964 Dr. Goodheart published an article describing how inhibited muscles could be restored to normal function through certain procedures designed to stimulate particular sensory end organs in an effort to correct neurological control features within the muscle and to provoke a more appropriate motor output from the central nervous system. Dr. Goodheart found that muscles could be monitored and that the response of test muscles to particular clinical procedures could reveal information about a broad range of body function. In effect, he found that these specialized clinical applications that grew out of knowledge of classical kinesiology—the study of body motion—could serve as a real time window into broad categories of body function. Goodheart went on to train many chiropractors and other health professionals in what he called Applied Kinesiology (AK). Among the select group of people who became “charter diplomates” in AK were chiropractors like Victor Frank, D.C., John Thie, D.C., and others who took muscle response testing in new directions often starting their own schools of study which now range far and wide and are practiced by virtually every type of health care professional, and many lay people interested in improving the function of the body. There are almost no energetic techniques that do not owe some measure of thanks to Dr. Goodheart for his ingenuity and creativity.

“Vial-based” techniques developed out of the notions of homeopathy, specifically the idea that water can be charged with an energetic imprint that it may hold for a very long time. Another influence on the development of vial-based techniques was the work of Dr. Raymond Rife. Rife may be among the best known doctors from the 1930's, 40's, and 50's who pioneered a field of study known as radionic medicine. This esoteric area of study involved the production of electronic apparatus that were thought to be able to conduct and transmit electromagnetic energy in frequencies that were believed to capture something of the essence of the real substance they were made to represent. In performing vial-based methods, these radionic instruments were used to imprint a carrier substance held in a small glass vial. The substance might be water, or combination of water and alcohol. There developed from this point forward in time a small industry based on the production and sale of such radionic instruments to healthcare professionals for uses that included the production of radionically prepared test vials. There was considerable pressure from federal regulatory agencies to suppress this industry as an example of medical quackery. Still the industry thrives, not in small part because there are many healthcare providers around the world who have found that vial-based techniques of energetic medicine are sometimes effective—often when conventional medical procedures are least effective.

It is the position of the inventor that until such time as a machine is produced which can read, and display the energetic signature of a previously manufactured radionically charged vial that no confidence should be given to the proposition that such vials actually do carry and radiate an energetic representation of anything besides the water, glass, and plastic that physically comprise the vial. It is even doubtful that an energetic signature of these actual substances is perceivable to a patient.

In order for a machine to duplicate a vial several things must be true. The machine must be able to sense the energetic signature that has been imprinted into the subject vial to be duplicated. The machine must then translate that energetic signature into either a digital, or analog electronic representation. The machine must then amplify, and transduce this signal in such a way as to energetically imprint the material that fills the target vial. If this is the case, and a specific and unique electronic signal has been sensed from the subject vial, it should be a very simple task to associate this unique electronic signal with the library of such signals for common substances. The name of this subject vial could easily be displayed on a screen, or readout. No one has yet done this, and the most likely reason for this is that it cannot be done—as we should expect if the vials carry no perceivable energetic signature.

It should be understood that the inventor does not take the position that these energetic techniques based on the use of radionically charged vials do not result in improvement of patients' health. It is the position of the inventor that the founders of these techniques made a clinical observation that they could perform an operation that resulted in an improvement in the patient's symptoms; but that they misapprehended what they had observed. As a result of this miss-take they fabricated systems of sometimes arbitrary treatment procedures, and a philosophy based on conclusions that are not supportable by any current means of evaluation. An adequate explanation of the observed efficacy of vial-based techniques may be provided by the simple explanation that these vials serve as nothing more than a metaphor, or mental placeholder for the practitioner performing such treatment.

The observed clinical phenomena interpreted to be evidence that radionically prepared vials transmit information to the patient are more elegantly explained by the explanation that the vials serve as a metaphorical point of focus for the practitioner to clearly frame intent which is then transmitted to the patient energetically through “Other Than Conscious” To “Other Than Conscious” communication (OTC).

The inventor also asserts that what are referred to by various treatment techniques as alarm points, reflex points, organ points, and meridian alarm points are also simply metaphorical concepts held in the mind of the clinician, and transmitted on an OTC/OTC basis to the patient during the process of performing the various steps of clinical evaluation that comprise the technique in question.

This explains why the developers of these various energetic techniques may use representational points that vary greatly from one energetic technique to another. In the study of NAET it is revealed that the organ alarm points for the liver and the gallbladder, as well as for the pancreas and spleen, are the exact opposite in position of those points representation in the treatment systems known as TBM, and NET. It is widely observed that the systems of treatment are effective to one degree or another. This supports the assertion that these “points” are nothing more than metaphorical mental placeholders for the practitioner and serve as a focal point for the practitioner's intent. It should be mentioned here that there are point-based systems of treatment, specifically classical acupuncture, for which objective investigation has established a relationship between particular locations on the body wall, and particular types of body function. Classical acupuncture appears to be the exception to the other point-based healing methods. Still, there are acupuncturists who feel that the point-based system of circuits is itself a metaphor for some energetic control system of the body, and a recent article in “The Skepical Enquirer”, Journal of PSICOPS, suggests a model of acupuncture that is frequency-based and solely related to energetic fields.

Prior Art

The disclosed invention offers a nearly limitless framework, grounded in rational principles that moves the science of energetic medicine to new levels. Prior methods grounded on a foundation of misperception and cloudy thinking have absolute limits to their development. Many of the methods discussed have remained virtually unchanged for over a decade. Progress requires leaving behind old notions that no longer stand the test of scrutiny. The following critique of prior art is offered with admiration, and respect to those whose contributions preceded the disclosed invention therapy.

Victor Frank, D.C. and Total Body Modification

Victor Frank D.C., developed and introduced Total Body Modification (TBM). This was a method that was based on both the use of radionically prepared vials, and body points—most of which represented the various organs and systems of the body. The teaching of TBM consisted of many separate protocols of procedure specific to various conditions, or complaints. The basic idea of TBM was that a symptom was produced when there was an energetic disharmony in the body that resulted in a compromise of the normal regulatory systems. A radionically charged vial would be selected through muscle response testing in which the presence of the vial in the patient's hand caused a reversal of a previously strong/weak test muscle. The practitioner would then continue muscle response testing until an alarm point was determined which countered the previously described weakness, thus establishing a relationship between the vial-produced weakness, and the organ point. Once this relationship had been established, the patient would hold the active organ point, and the doctor would perform tapping along the spine according to a selection of spinal levels thought to be associated with that particular alarm point in an effort to establish balanced neurological function. With this general rule established, a vast array of separate protocols for different conditions was created. If there was dysfunction in an organ such as the liver, the doctor might find that a vial representing alcohol, or hepatitis B virus countered the weakness generated when a contact has held over the liver. Vials representing infectious organisms were often used in the TBM protocols.

Each organ point had a corresponding sequence of vertebra levels that were to be adjusted with a chiropractic mallet that was performed to affect the process of harmonization. If the patient were allergic to a particular substance, that relationship would be shown by a weak muscle test when the patient held the vial, and then an organ point would be found which countered the weakness, suggesting an energetic link. The treatment process had many steps that would involve testing for and adding vials to the patient's hand. These vials might represent immunoglobulins, blood, histamine, or other substances. Each time a new vial was added, the process of finding an active alarm point, and treating vertebra sequences would be repeated until all steps of the protocol were completed.

In TBM, there was actually a third method of representing some feature of the treatment—hand gestures. There were certain gestures of the doctor's hands across the patient's body that were used to represent the relationship between various organs, or the direction of some process that was to be part of the treatment protocol.

Needless to say, TBM was a complicated technique involving alarm points, radionically prepared vials, and hand gestures. All of these aspects of treatment in TBM were simply a metaphorical way of communicating corrective information of one degree of accuracy or another to the patient on an “other than conscious” to “other than conscious” level (OTC/OTC).

The teaching of TBM emphasized the rote learning of these protocols, and spent little time in the discussion of physiology of disease processes. The intent of the practitioner was therefore incomplete with respect to the instruction he/she was attempting to deliver to the disorganized nervous system of the patient on an OTC/OTC level. This is one of the factors responsible for the inconsistency of success in applying TBM treatment protocols and that this criticism applies to virtually all other energetic techniques.

The present invention seeks to overcome the barriers placed by using metaphorically representational systems and to work with a direct method of introducing corrective information to the patient's control system, overcoming prior art inconsistency.

Devi Nambudripad, M.D., D.C., L.Ac. and NAET

In the 1980's, a chiropractor/acupuncturist named Devi Nambudripad developed a system of treatment for allergies that she named Nambudripad Allergy Elimination Technique (NAET). Dr. Frank's TBM allergy treatments were conspicuously close to NAET allergy treatment in execution. NAET treatment is simpler, with many of the steps Dr. Frank included in his method eliminated. One difference in the teaching of NAET as opposed to TBM was that, at least in the beginning of treatment of a particular case, there was a reasonably clear step-by-step protocol by which particular substances should be evaluated to see if they were functioning as allergens responsible for the patient's complaints. There was a group of ten basic substances representing the major nutrients, and more recently the introduction of a file called the BBF file, which was said to represent the organ systems of the body. Beyond this, the pathway by which to progress a patient through a course of treatment seemed much less clear. The practitioner was confronted with a seemingly endless catalogue of individual “allergen” substances, or at least what are purported to be energetic representations. The challenge was to search through that catalogue; more or less by trial and error, in hopes of finding the one which when treated would finally resolve the patient's complaints.

It is not unusual that a patient with a complex set of allergy responses might require many dozens of NAET treatments over the course of years to reach a reasonable level of therapeutic success. Cases in which the relationship of the complaint to an actual allergic response to some substance was less clear were even more confusing to manage. Not everything is an “allergy”.

In both the practice of TBM and NAET, failure or at least compromised success, results from the degree to which the attempt to input corrective information is ineffective, or confusing to the nervous system of the patient. This is because it is not clearly understood by the founders of TBM and NAET exactly what operational changes they are requesting of the autonomic control system of the patient they are treating.

It may also be that the tendency for temporary adverse responses to treatment with these methods also results from the degree to which the “Corrective Information” these methods offer is confusing, incomplete, or unclear to the patient's nervous system —pushing an already dysregulated control system further out of balance.

In TBM or NAET treatment, a vial is placed in the hand of the patient. Following this, there is a test of an indicator muscle for a change in strength, and then stimulation the spine with vertebral tapping. The process inputs a presumed stimulus, the presence of the vial, and attempts to observe patient response by way of a change in test muscle strength. With regard to specificity of the investigation, it must be asked, what question has been posed to the autonomic control system of the patient, and what corrective information has been uploaded to the patient. Clearly, it is not obvious from the teaching of these methods just what constitutes these informational factors. The vial itself provides no information.

When these other methods are performed there is a question inherent in the performance of the protocol, unspoken and usually unrealized by the practitioner which is communicated to the patient in an unspoken OTC/OTC communication. The unspoken question to the patient is, “Is the substance you are holding producing an allergic response?” in the case of NAET; or “Is the substance you are holding producing an energetic disharmony?” in the case of TBM. These questions are inherent in the practitioners.

When the corrective portion of any energetic protocol is performed, a command unspoken and unrealized by the practitioner, is uploaded to the patient by OTC/OTC communication. That command is that the patient's control mechanisms should no longer respond to the presence of the substance treated as if it were a harmful, or disruptive influence.

Scott Walker, D.C. and Neuro-Emotional Technique

The teaching of TBM involves many different course levels. The most advanced course level is the Questor, or Research level of training where various practitioners of TBM present original work. During one of the research level trainings in the 1980's a chiropractor named Scott Walker, D.C. presented a protocol in which emotional/psychological issues can be addressed. Dr. Walker discussed this with Dr. Frank, and they agreed that this work was related to, but fundamentally different from TBM. As a result of that conversation, Dr. Walker developed his own treatment protocols which he called NeuroEmotional Technique (NET). In performing NET Dr. Walker would find a negatively charged emotional experience in the patient's memory, and would have the patient hold the memory of that event in their attention while treatment similar to that in TBM was performed. The patient would hold a related organ reflex point while a sequence of vertebra was stimulated with tapping from a chiropractic mallet. In this sense, Dr. Walker was using the memory of the negatively charged emotional experience in the same way that Dr. Frank was using the energetically charged vials. This difference was necessitated by the fact that if the patient's complaint was caused by the way in which they were processing a stressful experience in their history, it would be necessary to determine the exact nature of that event, and through the protocol permit the patient to reprocess their response to the event more constructively. There would not be an opportunity to produce a series of vials that would cover every eventuality in this regard. For this reason, Dr. Walker developed a system of investigation that was essentially a binary decision tree in which the patient would be semantically confronted with various categorical questions, and the yes/no response to each of these would lead to a subsequent question until something known as the “snapshot” had been determined. This snapshot constituted the target event producing a disturbance for the patient.

In more recent years, Dr. Walker has changed his system of investigation, and now looks for more generic categories comprising disturbing experience to the patient rather than targeting very specific events. It appears that, at least in part, the reason for this was the understanding that particular events divined by the NET MRT process were not necessarily literal historically. They might be “emotionally real”, as opposed to “historically real”.

Dr. Walker introduced specificity of investigational information content to the process of assessing the patient, and moved in a direction away from the use of metaphorical representation. Less emphasis was placed on the importance of the specificity of communication with the patient's autonomic control system, or that such communication could occur on as literal a level as conscious verbal conversation. This is evidenced by the treatment portion of NET. In this portion of treatment, which is referred to in NET as poising the patient, there is no specific corrective semantic input. The patient simply holds the “snapshot”, or the more generic category of disturbing emotional experience in their attention while an organ reflex point is held, and specific spinal levels are stimulated with a chiropractic mallet.

### SUMMARY OF THE INVENTION

Neuromodulation therapy (NMT) takes the position that the patient's autonomic control system is directly accessible through the use of carefully applied muscle response testing, or MRT. MRT opens a window through which the status of the autonomic control system (ACS) can be accurately, and thoroughly assessed. MRT is the preferred methodology because it is a clinically friendly tool in the hands of a practitioner competent in its use, and because no special instrumentation is required to perform the evaluation. Other methodologies may also be used, including electronic instrumentation based on galvanic skin response, or scalar wave transmission as an indicator of ACS response. For those practitioners who have difficulty developing facility with MRT, other means may provide a more reliable tool of investigation.

In the practice of the invention, performance in the autonomic control system is modulated with a combination of verbal and/or non-verbal corrective commands and statements. This process is further augmented and reinforced with percussive, or other stimulation of vertebral segments, specific breathing patterns, and other sensory stimuli. The invention is based upon widely accepted neurophysiological models. Muscle response testing is used to access, and assess the autonomic control system, and to modulate the performance of autonomic control functions with specific semantic reprogramming and debugging scripts.

Neuromodulation therapy can be used to treat a wide variety of illnesses that are provoked by faults in the function of the autonomic control system. This list includes allergies, chronic degenerative diseases such as arthritis, multiple sclerosis and other demylenating diseases, Crohn's disease, IBS, and gastric reflux disease, infectious disease processes, autoimmune diseases, acute and chronic musculoskeletal conditions, addictions, emotional, psychological and sensory/motor neurological disturbances.

Muscle response testing (MRT) is the preferred method to access the autonomic control system of the patient. The muscle response test is a form of communication with the patient's autonomic control system. The conscious mind, regardless of the level of education, or intelligence of the patient is generally ignorant to vegetative functions that are controlled by the autonomic control system. Attempts to ascertain the information needed to treat the patient through a two-way verbal conversation are largely useless. Muscle response testing affords the opportunity of communication with the autonomic control system, but that communication is limited on the patient's side to the binary yes/no response we attribute to the fluctuations in strength noted in the selected test muscle. This method of communication still affords great opportunity for an in depth assessment of the status of the patient, dependant on the precision and clarity of the query/command statements that are directed at the patient.

In the preferred methodology of the disclosed invention clinical algorithms are used for other than conscious to other than conscious (OTC/OTC) communication. These algorithms are special sets of interrogative statements used to ascertain characteristics of the ACS and corrective commands or directive statements used to inform the ACS of a more correct mode of behavior. These algorithms are referred to as “pathways” and various sets of interrogative and directive statements make up each of numerous pathways—each of which addresses a particular aspect of function of the body. The purpose of bringing to mind and speaking these semantic algorithms permits the practitioner to form and shape a mental picture of the meaning of the pathway statement.

Corrective utilization of the invention is a matter of assessing where neurological dysfunction is occurring, the nature of that dysfunction, and then correcting this either with commands that result in a more functional distribution of information in the nervous system, the elimination of faulty information, and/or in the alteration of some ongoing process of neurological facilitation, or inhibition.

An aspect of the clinical skill to perform the disclosed methodology is the acquired proficiency to form and shape a concise and unambiguous mental picture of each step of a clinical pathway and to project that thought image to the patient. The development of this skill is directly related to the repetition of practice and the intensity of focus and concentration on the act.

### FIGURES & TABLES—

The FIGURE and tables referenced are a best method outline of pathway interrogation, and are for facilitation in the understanding of the art and practice of the invention and should not be considered limiting or restrictive.

FIG. 1—A depiction of the preferred mode of MRT

TABLE 1—Pernicious Synaptic Patterns

TABLE 2—The Sensory/Motor Pathway (SMP)

TABLE 3—The Allergy Pathway

TABLE 4—The Infectious Agent Pathway

TABLE 5—The Exogenous Analog Pathway

TABLE 6—The Toxin Pathway

TABLE 7—The Morphic Fields Pathway

### DETAILED DESCRIPTION OF THE INVENTION—BEST MODE

MRT

Muscle response testing (MRT) may utilize any muscle in the body, and testing may be performed in any position that is convenient. O-ring testing, and other such styles of muscle testing are all valid, and there may be application for these in the course of practicing the invention. The leg length tests taught in Activator technique, Direct Non-Force Technique (DNFT) and other methods are all MRT approaches based on OTC/OTC communication and could be used in practicing the invention. Arm length testing may be used in the same way.

Whether the positive response is interpreted as weak, or strong may be cosnidered arbitrary. To practice the invention, the practitioner must principally be clear on what they know a response to mean. If in doubt, the patient or subject to be treated may be queried with “show me a ‘Yes’ response.”

The practitioner who has clarity in the method he/she has been trained in will communicate these ground-rules of communication automatically to the patient, and without conscious awareness of the patient, or perhaps the practitioner. Obviously, any confusion in the mind of the practitioner about the MRT procedure will contribute to opportunity for error in getting a meaningful response from the patient.

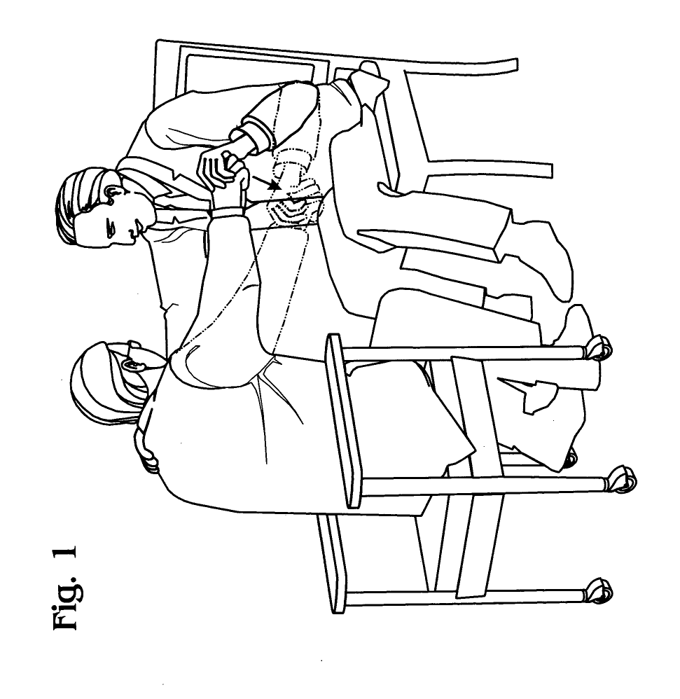

The practitioner will perform many muscle tests in the course of the day whereas the subject in most cases will only be treated once in a day. Therefore, the setup that is chosen for the muscle response testing should be one that ergonomically favors the practitioner. The position that its preferred in practicing the invention is one in which the subject and practitioner face one another in a seated position as depicted in the FIGURE. FIG. 1 The subject will extend their arm at right angles from the shoulder with their hand in the light fist position. The subject will use the anterior deltoid muscle exclusively to resist. The subject's hand is rotated to a palm down position. The practitioner grasps the face of the subject's fist with an open palm. The practitioner's humerus is perpendicular to the floor. The procedure at the moment of effort in the muscle response test requires that the practitioner ask the subject to resist while the practitioner pre-loads a little tension into the subject's arm, and then loads a moderate level of resistance into the subject's arm with a smooth, short, steady stroke as depicted in the FIGURE. This is extremely easy on the practitioner, and is not overly fatiguing for most subjects. The practitioner should alternate test arms frequently to avoid fatigue in the subject.

The preferred subject position for MRT is with the subject on a task chair with a swivel base, and the back of the chair removed. FIG. 1A chair that has armrests is more comfortable and makes rotation of the subject during the course of evaluation and treatment easier. The chair has no back so that the subject's spinal area can be easily accessed. In this position, a full physical examination including orthopedic and neurological testing may be performed. Most physical examination tests may be performed in either a standing, or seated position; or slightly modified to be performed in these positions. This position is highly efficient in the management of subject flow. Once the required level of physical examination has been performed in the sitting position, the practitioner may perform the muscle response testing and treatment required on that visit. The course of a particular visit may require multiple muscle response tests and treatment. When the practitioner and subject are seated facing one another as described, this can be done very quickly and easily. When special circumstances dictate otherwise, whatever positional arrangements for testing and treatment appropriate to that situation may be used.

When performing MRT, the practitioner is concepting either a statement or question and looking for a change in muscle activity from some established baseline. As discussed earlier, the MRT response is always a response to the OTC/OTC communication of practitioner and subject.

Whatever method of muscle testing is used, whatever position the subject is tested in, the primary consideration is that the practitioner projects his/her intent with focus, clarity and comprehension of subject material, and with the greatest possible power of projection of that intent. This is not so much a matter of effort as it is practice to find the mental posture to assume while engaging in MRT.

The Source of MRT Information

Some students of energetic medicine, and some teachers of energetic medicine techniques may have different assertions about the information that is gathered by muscle response testing. Some take the position that muscle response testing somehow taps into a great reservoir of universal knowledge and involves a flow of information at an OTC/OTC level. That belief is essential to David Hawkins writing on the subject of MRT and discussed in his books, Power Versus Force, and The Eye of the I. Others take the position that muscle response testing works on a spiritual level. They might argue that the answers come from some spiritual entity, either benign or malignant, depending on one's point of view

The source of the information gathered through MRT is largely irrelevant to the practice of the inventions, so long as the information is acquired by the practitioner to permit treatment.

What Information can be Determined by MRT

In the practice of the disclosed invention, MRT queries the autonomic control system of the subject, and is generally limited to what information the ACS has to reveal. The subject's ACS is, almost by definition, dysfunctional. Otherwise the subject would not be a candidate for treatment. At its best the ACS has limits, just as its conscious nervous system counterpart has limits, to how much processing it can perform in a period of time and how much information it can maintain an awareness of at any given moment.

Those limits may be different from one subject to another, and from one encounter to the next. It is reasonable to infer that the more simple, direct, unambiguous the question, and the more accomplished the practitioner is in forming, shaping, and projecting his/her intent the greater the probability of a reliable answer.

The practitioner may find that a particular line of questioning comes to an end, but that a subsequent exam reveals something more. For example, with treatment of the Allergy Pathway, Table 3, the practitioner asks the ACS to consider all allergenic triggers it has recorded in memory that can provoke an immune system response. The ACS may not have registered all allergens in its exposure history when interrogated. If this is the case, on subsequent evaluation it will be found that the subject may have improved, but that some allergenic triggers are still active. The reason for this is that if these still-active allergenic triggers were not within the window of awareness generated at the previous treatment, they were never part of the group of allergens that were the subject of that treatment and, therefore, escaped correction. Such findings do not represent error in the procedure, but reflect the limitations of the human nervous system that the practitioner must be aware of when practicing the invention. This will be more, or less of a problem from one subject to another. When it is a problem, solutions include making a semantic reference to particular substances to be sure it is within the ACS's field of view during treatment. The practitioner may wish to have a lists of specific substances—nutrients, toxins, heavy metals, etc. as a reference to use in identifying particular suspected allergens. The same thing can be done using lists of body tissues or sensory end organs as the Sensory/Motor Pathway is performed.

Other factors that influence the accuracy of the muscle response test have to do with the clarity and depth of understanding of the examiner of the issues being investigated.

The greater the clarity and depth of understanding the examiner possesses the more accurate and valid will be the response from the subject. The critical factor is the clarity of understanding in the examiner, not in the subject. It is not necessary that the subject have an understanding on a conscious level of the questions that are posed during MRT.

To effectively practice the invention, the practitioner should first learn the query/command statements well enough that they are recalled quickly and accurately, and with clear comprehension. Failure to achieve proficiency will likely result in unclear communication to the subject's ACS and treatment will either fail outright, be sub-optimal, or may even confuse the ACS into further inappropriate function.

With proficiency and use of the pathway statements, eventually each statement of the pathway will have a unitary meaning, rather than simply being a string of words. The practitioner will achieve a level of familiarity with the clinical pathways such that if the practitioner uses a flip chart, or PDA display of the pathway he/she will produce an instantaneous iconographic representation of it by just glancing at the written representation of the query/command.

Treatment of Pernicious Synaptic Patterns—PSPs

PSP's are defined as a dysfunctional distributed network of erroneously recorded data patterns in the central nervous system. Specifically, it is the persistence of a dysfunctional and inappropriate network of such data of sufficient complexity and structure to function as a set of dysfunctional instructions, or mini-programs. These entities are capable of producing harmful chaotic behavior within the nervous system, and compromising otherwise appropriate neurological functions. The symptomatic expression of such entities is dependent upon the way in which the pernicious synaptic pattern is structured to interface with, and potentially interfere with some particular functional aspect of the nervous system. Consequently the influences of PSPs on any somatic, emotional, or intellectual process may cause almost any imaginable symptom.

The persistence of data in the brain, the lasting trace of previous experience be it external stimuli or internal processing of information may be referred to as memory. It is generally agreed that this memory is physically represented by patterns of synaptic connection, much like the pattern of zeros and ones in a computer is a physical representation of information. The proposition that synaptic patterns in the CNS can influence function before voluntary activity even begins is well documented. A common manifestation in this phenomenon can be seen in the contemplation of eating which can initiate motor responses in the gastrointestinal system.

When data is recorded inappropriately the resulting units of information become what is referred to as “pernicious synaptic patterns”. Such patterns, being by definition unsuccessful or inappropriate recordings of data, could exist as such disorganized and simple traces in the nervous system as to be unable to influence any neurological function. However, when such pernicious synaptic patterns have sufficient complexity and organization to function as a program in the nervous system, and to the extent that they may interact with normal voluntary or involuntary nervous system processes, they may result in degradation of neurological performance.

The disclosed therapy maintains that PSPs can be of a sensory, motor, or an emotional nature. PSPs are believed to be distributed networks of synaptic connection, rather than being confined to a specific anatomical area. PSPs that have the greatest influence on function may be presumed to reside primarily in areas of the brain associated with planning and execution of actions.

Corrective application of the invention is a matter of assessing where neurological dysfunction is occurring, the nature of that dysfunction, and then correcting for this either with commands that result in a more functional distribution of information in the nervous system, the elimination of faulty information, and/or in the alteration of some ongoing process of neurological facilitation, or inhibition.

PSPs exert effect to the degree that they are attached to some particular area of memory, or function. The preferred method of accessing particular groups of functionally related PSPs involves activating the related memory areas of the nervous system by various means. PSPs associated with particular motor or sensory experience and activity may be engaged by having the subject assume a particular posture, motion, or experience a particular sensation. PSPs associated with a particular area of physical injury, or disease may be engaged by mild physical provocation of the area.

Tapping, stretching, pressing, or specifically posturing a physical area suspected to be related to stored PSPs while the query for PSPs is performed will often bring more PSPs to a level of ACS awareness. PSPs associated with more complex experiences, or relationships may be engaged semantically by asking the subject to consider their recollection of such topics. Suggesting particular context, or relational considerations that may interplay with the target experience may further enhance this. The end result of such activities on a clinical level with the subject will be to bring to a level of awareness the PSPs that have been successfully referenced.

In the practice of the invention, the assessment of PSPs involves questions that specify the existence of active, inactive, and latent PSPs. This is simply a distinction of the relative degree to which a PSP is engaged or activated and therefore the degree to which it is currently affecting physiology. An active PSP is fully engaged and currently influencing physiology. An inactive PSP is not engaged at the moment the MRT query is posed, so unable to influence physiology at the time of the MRT. The inactive PSP is at a high enough level of awareness that it is very likely to be engaged at some future time. A latent PSP is a pattern that has the potential to become engaged given the appropriate triggering experience, but otherwise exists at a more remote level of awareness than the inactive PSP. These categories for PSPs are of value in that by specifying characteristics of the memory storage of the PSPs, it may favor the efficiency by which the ACS is able to recognize PSPs. This makes more PSPs the subject of corrective instruction.

The failure of normal neurological controls is the result of some interference to the optimal expression of neurological programming that expands from the initial DNA database of the individual. Under the invention theory, it is maintained that the autonomic control system is a self-regulating, plastic and intelligent entity that will take appropriate corrective measures once it has been given awareness of it's error.

With regard to PSPs, the correction causes the autonomic control system to identify and quantify relevant PSPs, recognize the fact that they are compromising optimal function of the system, and that the solution to this problem is to disorganize these pernicious synaptic patterns.

A best mode outline of treatment for PSPs is described below. The statements or algorithms provided are as listed in Table I with directive instruction to facilitate use and understanding of the practice of the invention.

In the application of the invention, the practitioner interrogates the subject to be treated, preferably using MRT as described above with the following queries:

1. “Are there active, inactive, or latent PSPs associated with this complaint?” The use of semantic cues, position, posture, muscle challenge, and other methods taught to provoke PSPs to a level of awareness may facilitate treatment. If no, check MRT, and cognitive valuation for confirmation. If yes, proceed to the following sub-menu.

a. “Is there any fault in cognitive valuation compromising the perception of the full spectrum of PSPs related to this condition?” If no, go to next question. If yes, proceed to the following sub-menu:

i. “Is any level of the ACS aware of correct cognitive valuation with regard to this” If no, check MRT. If yes, proceed to the following question.

ii. “Are all levels of the ACS aware of correct cognitive valuation with regard to this” If no, check MRT. If yes, proceed to the following question.

iii. “Would it be of benefit to treat with the intention of making all levels of the ACS aware of correct cognitive valuation in this regard?” If no, check MRT. If yes, proceed to the following question.

iv. “Would it be of benefit to treat with the intention of correcting all faults in cognitive valuation at this time?” If no, check MRT. If yes, proceed to the following question.

b. “With regard to the number of active, inactive, and latent PSPs is the number 10 to the Xth?” To quantify PSPs start with X=10 and work up by multiples of 10 (next number in this series is 100, then 1000) until a “no” is returned. Then cut the previous interval by halves until you arrive at a number that tests affirmative. If the first cut is of the previous interval in half, and this was too low, go up by half of the interval toward the number that first gave a “no” until a correct number is determined. Accuracy to the whole number at the power level is adequate. It is seldom necessary to spend the time to narrow this down further. The practitioner should not project expectations for the quantity of PSPs expected. To do so may “stop down” the field of view. Realize that the effect of ancestral morphic fields may account for a significant number of PSPs identified. The practitioner should be aware of the way ancestral morphic fields inform present function.

c. “Would it be of benefit at this time to treat with the intention of making all levels of the ACS fully aware of the identity and existence of these PSPs, that they are producing harmful behavior, and that they must be disorganized to restore normal function?” If yes, treat spinal points with breathing cycles and proceed to the next question. In the preferred method, spinal points are treated by a series of rapid impacts along the spinal column as with a chiropractic tapper or impact tool to flood the neural pathways with stimuli.

d. Perform serial rechecks within a visit session using the methods disclosed for provoking the subject to awareness until no further PSPs are evoked, or satisfactory symptom improvement is achieved.

e. A future vigilance statement may be used along the order of: “Would it be of benefit to treat with the intention of causing the ACS to maintain an awareness for any future presentation of pernicious synaptic patterns (related to this condition), and upon recognition to cause the ACS to recognize the identity and existence of these pernicious synaptic patterns, the fact they are causing harmful behavior, and that they must be disorganized to permit a return of normal function?” If yes, treat spinal points with breathing cycles.

Treatment of Sensory/Motor Dysfunction

The sensory/motor pathway is a clinical algorithm consisting of query and command statements for the purpose of assessing and making corrective modification in a dysfunctional sensory/motor control loop responsible for some aberrant physiology. This pathway addresses dysfunction at each of the major constituents of the sensory/motor control process. It assesses and corrects problems in the setting of the various tissue embedded sensory endorgans, the processing of such afferent signals in the central nervous system, the central nervous system processing by which a motor response to these incoming signals is determined, and the threshold of stimulation for motor endorgans of the muscles and secretory tissues that receive the resulting motor signals.

This clinical algorithm has a broad range of application that extends from the treatment of chronic pain and paresthesia, to dysfunction in gastrointestinal motility and performance, to the control of hormonal production and release, for example by the application of this sensory/motor pathway to hypothalamic control of pituitary function. This pathway, as with others, may be used effectively in conjunction with other clinical pathways for greater effectiveness.

A best mode outline of treatment for sensory/motor dysfunction is described below. The statements or algorithms provided are as listed in Table 2 with directive instruction to facilitate use and understanding of the practice of the invention.

1. “With regard to the (specify the sensory/motor complaint, dysfunction, tissue, organ, or system) is there any fault in sensory/motor control causing, or contributing to this condition (dysfunction of tissue, or system, etc.)?” Alternatively, a specific area may be provoked mechanically, or ACS attention reinforced by contact of the area during MRT. If no, proceed to another area of investigation. If yes, proceed to the following sub-menu to determine any concomitant involvement of other pathways:

a. “Is there any role of an infectious agent causing, or contributing to this condition?” If yes, the infectious agent pathway will require treatment at some point.

b. “Is there any role of an allergic phenomena (endogenous, or exogenous allergen) causing or contributing to this condition?” If yes, the allergy pathway will require treatment at some point.

c. “Is there crossover autoimmune phenomena between infectious agent and body tissue; or an endogenous/exogenous allergen and body tissue?” If yes, treatment of the crossover autoimmune phenomena will be required. It is possible that the body may retain autoimmune behavior triggered by an infectious agent that is no longer present, or an allergenic trigger that is no longer active.

2. “Is there any fault in cognitive valuation with regard to any aspect of the condition?” If no, proceed to the next question. If yes, proceed to the following sub-menu:

a. “Are all levels of the ACS fully aware of correct cognitive valuation of all aspects of this condition?” If yes, proceed to “b”. If no proceed to the following sub-menu:

i. “Is any level of the ACS fully aware of correct cognitive valuation of all aspects of this condition?” If yes, proceed to the following question. If no, investigate the possible cause.

ii. “Would it be of benefit to treat at this time with the intention of making all levels of the ACS fully aware of the correct cognitive valuation of all aspects of this condition?” If yes, treat spinal points with breathing cycles and proceed to the next question. Consider that in some cases it may be necessary to investigate specifically the nature of the fault in cognitive valuation. Also consider that there may be forks in the pathway where specific issues of fault in cognitive valuation appear—suggested by apparently inappropriate MRT responses.

b. “Would it be of benefit to treat at this time with the intention of correcting all faults in cognitive valuation of the condition?” If yes, treat spinal points with breathing cycles and proceed to the next question.

3. “Are there active, inactive, or latent PSPs associated with this complaint?” If no, proceed to the next question. If yes, proceed to the following sub-menu, and also be aware that PSP issues may present at any fork in a clinical pathway, suggested by inappropriate responses to MRT.

a. “Is there any fault in cognitive valuation compromising the perception of the full spectrum of PSPs related to this condition?” If no, proceed to the next question. If yes, proceed to the following sub-menu:

i. “Is any level of the ACS aware of correct cognitive valuation with regard to this?” If yes, proceed to the next question.

ii. “Would it be of benefit to treat with the intention of making all levels of the ACS aware of correct cognitive valuation in this regard?” If yes, treat for this statement, check for correction, and proceed to the next question.

iii. “Would it be of benefit to treat with the intention of correcting all faults in cognitive valuation with regard to this?” If yes, treat for this statement, check for correction, and proceed to the next question.

b. “With regard to the number of active, inactive and latent PSPs is the number 10 to the Xth?” To quantify PSPs start with X=10 and work up by multiples of 10 (next number in this series is 100, then 1000) until you get a “no”. Then cut the previous interval by halves until a number that tests affirmative. Accuracy to the whole number at the power level checking is adequate. Doing a bracket query, and in establishing over and under limits to this number will effectively quantify the PSPs. It is seldom necessary to spend the time to narrow this down further.

c. “Would it be of benefit at this time to treat with the intention of making all levels of the ACS fully aware of the identity and existence of these PSPs, that they are producing harmful behavior, and that they must be disorganized to restore normal function?” If yes, treat for this statement, recheck for correction, and proceed to the next question.

4. “Is there any fault in the tagging of afferent stimuli relating to the perception of this condition?” If no proceed to the next question. If yes proceed to the following sub-menu:

a. “Is this afferent specific?” If no, the fault is global and involves all SEO afferents, so proceed to the next question. If yes (afferent specific), proceed to the following sub-menu:

If there is a fault in the tagging of an SEO afferent when the CNS receives the afferent for processing the result may produce fantastic symptoms. A stretch receptor of fascia, perineurium, or other tissue may be the subject of inappropriate tagging and this may produce such robust paresthesia that a disk lesion might be expected, yet correction of the tagging fault may produce instantaneous resolution of the apparent sensory deficit. Always check closely for tagging faults when treating the SMP. MRT query to find the appropriate tissue and the particular afferent of that tissue that is the subject of inappropriate tagging, and proceed to the following question. (eg. There may be pain in an area due to the inappropriate tagging of the sensory stretch afferent from fascia tissue; meaning that fascial stretch afferent data is being improperly interpreted as pain.) Corrective statements would then be made appropriate to the indicated specific tissue(s) and the afferent(s) of involved tissues. If there were several such faults, each could be identified in sequence and the ACS asked, “Can you register this fault?”. Once all such faults had been identified they could collectively be the subject of the treatment statements that follow. This specificity in identifying tagging faults facilitates the ACS to identify the problem areas to be addressed.

b. “Are all levels of the autonomic control system fully aware of appropriate tagging protocol for the indicated stimuli?” If yes, skip to next question. If no, proceed to the following sub-menu:

i. “Is any level of the ACS fully aware of appropriate tagging protocol for the indicated stimuli?”

ii. “Would it be of benefit at this time to treat with the intention of causing all levels of the ACS to be fully aware of appropriate tagging protocol for these stimuli?” If yes, treat spinal points with breathing cycles, confirm correction, and proceed to the next question.

c. “Are inappropriate tagging processes relative to the indicated stimuli occurring at (check for the involved nervous system site which will usually be amygdala, basal ganglia, or reticular activating system)?” If yes, proceed to the following sub-menu:

i. “Would it be of benefit at this time to treat with the intention of correcting all inappropriate tagging of afferent stimuli occurring at (specified site)?” If yes, treat for this statement, confirm the correction, and proceed to the next question.

5. “Is there a fault in any sensory end organ (SEO) contributing to this condition?” If no, go to the next question. If yes, go to the following sub-menu:

a. MRT query for the indicated tissue and sensory end organ. Investigate structures supportive to the tissues that are registering the problem. (eg. check structures associated with the vasculature of the inner ear when there is a problem with hearing.) Check deep, as well as superficial structures, (eg. disc annulus pain/stretch receptors with back pain.) If checking visceral function/complaint, concept through the anatomy that may be contributing to the condition (eg. muscular layers, capsules of viscera, fascia supporting the structure, etc.).

b. MRT query for inappropriately high/low threshold of sensitivity. For example: “Regarding the fascial stretch receptor, is this receptor set at an inappropriately high/low threshold of sensitivity?”

c. MRT query for the presence of inappropriate CNS level facilitation, or inhibition of the afferent from this sensory end organ. For example: “With regard to (the indicated SEO) is its afferent being inappropriately facilitated, or inhibited?”

d. MRT query for the CNS anatomical site of inappropriate facilitation, or inhibition of the afferent.

i. The vast majority of instances will reveal that the CNS site is the thalamus (T), hypothalamus (HT), or hippocampus (HC). If not, consider the amygdala as a next likely choice.

ii. MRT query from the general to specific to find particular CNS processing sites. Eg. CNS—Midbrain nuclei—Thalamus. Generally, when any tissue sensory end organ is being inappropriately facilitated, or inhibited that this is occurring at the thalamus, hypothalamus, or hippocampus; and sometimes the reticular formation. Less frequently, cortical level of involvement.

e. Continue finding SEO faults and associated faults in the CNS processing of each SEO. Correct faults with the following corrective statement: “Would it be of benefit to treat at this time with the intention of resetting (the indicated SEO) to a higher/lower threshold of stimulation, and to stop all inappropriate facilitation/inhibition at the (specified CNS anatomical site), consistent with optimal function?” Recheck for correction, and repeat this process until all sensory end organ function tests clear.

f. Stacking of sensory end organ corrections: With experience and practice it is possible in most cases to simply go through the MRT procedure to identify all sensory end organ faults, and the attendant levels of inappropriate CNS facilitation, or inhibition thereby accumulating a catalogue of corrections to be made by the subject's ACS. After each such fault is determined ask, “Can you register this fault?” If the ACS indicates it is able to register a series of such faults then it is possible to continue until all sensory end organ fault patterns have been identified. At that point the corrective statement is made. eg. “Would it be of benefit at this time to treat with the intention of resetting all indicated sensory end organs to optimal sensitivity levels, and to stop all inappropriate facilitation and inhibition at the indicated CNS processing areas.”

g. Once the sensory end organ path tests clear to the semantic query, the process can be repeated if necessary for greater specificity. The practitioner will use his/her hand, or have the subject use his/her hand to isolated specific area during the query process. The hand may apply pressure to further isolate, stimulate, activate, or specify a particular tissue and sensor.

h. Placing the subject in a position that stresses particular tissues during the query process can further enhance the process of investigating sensory end organs. Attempt to MRT in this stressed position.

i. A further enhancement to the investigation of sensory end organ status may be performed at any point in examination. It may be done when SEO function is first assessed, after going through the SEO correction step, or after the entire SMP is completed and symptoms are still noted. Have the subject get up, and move about the room with particular attention to moving in a way that engages the tissues that are being investigated. When the subject moves about, the nervous system has an opportunity to reevaluate sensory end organ status and further improvement may be found at this point retreating some of the same SEO's. The subject, at this stage of the treatment, may also be able to move further through a range of motion and in so doing engage other types of sensory end organs, or more of the same type of sensory end organ that were not triggered in earlier stages of the examination. This provides for an opportunity to make a deeper correction at the sensory end organ level.

6. “Is there any fault in central nervous system processing causing, or contributing to this condition?” If no, proceed to the following question. If yes, proceed to the following sub-menu:

a. MRT query to see if there is a fault in the processing of an afferent from the body, an efferent to the body, or a fault in intra-CNS traffic. Faults in intra-CNS traffic will be the most common and extensive functional error in the category of CNS processing.

i. If this is a fault of the processing of an afferent/efferent determine if there is inappropriate facilitation, or inhibition (and check for excess, or deficiency of either process), the anatomical site of this fault (nearly always at thalamus, hypothalamus, or hippocampus; but possibly at cortical, or other areas), and treat to correct this. This is found far less often than intra-CNS processing faults. Be aware that such faults may apply to the various SEOs of the tissue, as well as motor efferents. It may also apply to motor efferents to endocrine tissue, eg. motor efferents from the hypothalamus to the anterior pituitary—often of great importance in hormonal issues.

ii. If this is a fault of intra-CNS processing, determine the origin/target sites if the issue is facilitation/inhibition of a pathway between two processing areas (for example, facilitation from reticular formation to hypothalamus—a common pattern). Intra-CNS faults involving sensory perceptions from peripheral tissues often involve a pathway with its origin at the reticular activating system and a target of T, HT, or HC. Begin the investigation by going from the general to the specific in your query: eg. cortex-frontal cortex-more specific site within frontal cortex, or midbrain nucleus-hypothalamus. Determine if there is more than one target site. Determine if there is inappropriate facilitation/inhibition (and check for excess, or deficiency of either process), or routing and treat to correct this. If there is an error in routing, check to see that all areas of the autonomic control system are fully aware of correct routing for this traffic. Determine if there is simply inappropriate facilitation/inhibition relative to some specific function occurring at a particular CNS processing nucleus.

iii. Common sites of intra-CNS processing for sensory issues include origin at RAS, and target of T, HT, or HC. Secondary path may exist in which the target is now the origin, and participates with another processing site. This may be another forebrain nucleus eg, T-HC; or it may involve a path from a forebrain nucleus to one of the cortical areas.

iv. If the presenting problem is not primarily a sensory issue then the intra-CNS issues may not involve the RAS, and may be centered more on paths between various cortical areas, and between cortical areas and forebrain nuclei. Consider the possibility of cerebellar involvement as it is considered a repository of templating of much motor processing.

v. Repeat this process until all intra-CNS processing tests clear.

7. “Is there any fault in sympathetic, or parasympathetic processing causing, or contributing to this condition?” If no, proceed to following question. If yes, determine whether sympathetic, or parasympathetic and proceed to the following sub-menu:

a. Determine whether this is a fault in sympathetic thoracolumbar, or cervical ganglia outflow, or cranio-sacral parasympathetic outflow. Think through the type of problem to see what is reasonable. If the subject has chronic diarrhea, consider facilitation within pelvic parasympathetic ganglia. If there is migrainous headache, consider a fault in cervical sympathetic ganglia innervation to vasculature to the brain.

b. Determine whether this is a problem of facilitation, or inhibition (check for excess, or deficiency of either process).

c. Determine whether this is a problem primary to the indicated autonomic ganglia, or is the result of a higher-level neurological dysfunction. Generally this tests as primary to the autonomic ganglia involved. If it is the result of higher CNS influences construct a corrective statement to resolve that influence, eg. “Would it be of benefit to treat at this time with the intention of stopping all inappropriate facilitation of sacral parasympathetic ganglia at the hypothalamic (HT) level.”

d. Construct an appropriate treatment statement and correct for this, recheck for successful correction, and proceed to the next question. For example, “Would it be of benefit to stop all facilitation of sympathetic efferents from the stellate ganglia (or, a more general statement referencing any involved sympathetic ganglia) to muscle wall of cerebral vasculature structures consistent with optimal function?”

8. “Is there any fault in motor processing causing, or contributing to this condition?” If no, proceed to the next question. If yes, proceed to the following sub-menu:

a. MRT query to see if this fault involves inappropriate facilitation/inhibition (Check for excess, or deficiency of either process).

b. MRT query for the CNS anatomical site of this fault.

c. MRT query to see if this involves motor efferents to muscles in the area of complaint, whether it is global to these muscles, or specific to agonists/antagonists.

d. Treat with an appropriately structured corrective command specific to your findings. For example, “Would it be of benefit to treat at this time with the intention of stopping all inhibition of motor efferents to antagonist muscles in the area of complaint?” (As a matter of convention “agonist” muscles will be considered those muscles in the area of complaint that are hypertonic and “antagonist” will refer to muscles which function as the kinesiological antagonist to those muscles.)

e. Repeat this process until all CNS processing tests clear.

9. “Is there any fault in a motor end organ causing, or contributing to this condition?” If no, proceed to the next question. If yes, proceed to the following sub-menu:

a. MRT query to determine if this is relative to a motor end organ of muscle, endocrine gland, exocrine gland or secretory skin structure. If this involves muscle determine if it is muscle in the area of complaint, if it is global, or specific to agonist/antagonist.

b. MRT query to determine if the motor end organ is set at an inappropriately high/low threshold of stimulation.

c. Treat with an appropriately structured corrective command specific to your findings. For example, “Would it be of benefit to treat at this time with the intention of resetting the indicated motor end organ of dermal sweat gland to a high threshold of stimulation consistent with optimal function?”

d. Repeat this process until all motor end organ function tests clear.

10. “Is there any other fault in autonomic regulation causing, or contributing to this condition?” If yes, MRT query as appropriate to determine what this is, and treat appropriately.

Treatment of Allergy Dysfunction

One of the processes by which allergies occur is a mistake of data processing. The process of homeostasis requires a system by which the body can control its internal chemical environment and screen for substances that might be harmful. This task is the responsibility of the immune system and the elements of the nervous system and energetic control systems that regulates immune function (the ACS). For some subjects who suffer from allergies this process has suffered from some level of confusion. In these subjects, the immune system and the regulatory agents associated with it make an error in which some innocuous substance is recognized as a threat to the internal environment of the body. This misinterpretation results in the immune system responses directed against that innocuous substance that produce the symptoms of allergy.

The allergy pathway is a clinical algorithm that defines the various components that constitute the allergy phenomenon. This clinical pathway is structured to permit the ACS to have an accurate awareness of the identity and existence of the substances to which it is producing the allergic response, to identify the location and nature of faulty neurological processing whereby the allergen is misidentified as a danger to the body, to correct various levels of confusion by which body tissues may be drawn into allergy physiology, and to address any contributory faults in sensory/motor processing, and PSP formation. The allergy pathway is used to correct allergic immune system responses to exogenous substances; but it is also used to correct the body's allergic response to its own tissues that occurs as part of many chronic degenerative conditions.

A best mode outline of treatment for allergy dysfunction is described below. The statements or algorithms provided are as listed in Table 3 with directive instruction to facilitate use and understanding of the practice of the invention.

1. “With regard to the referenced symptoms/condition, are there (Or, in the clear, “Is any level of the ACS aware of . . . ”) any inhalant, ingestant, contactant, or injectant substances (exogenous allergens) in your exposure history which are capable of behaving as allergenic triggers? (“ . . . causing or contributing to this condition?”) Always check, “With regard to the referenced symptoms/condition, are there (Or, in the clear, “Is any level of the ACS aware of . . . ”) any body tissues, body chemicals, or breakdown products of the body (endogenous allergens) which are capable of behaving as allergenic triggers (“ . . . causing or contributing to this condition?”) If no, proceed to investigate other pathways. If yes, proceed to next question.

Note that, clinically, it is usually preferable to treat exogenous and endogenous allergens at separate visits. Conditions may exist in which it is preferable to treat a specified allergen trigger(s), or class of allergens on a particular visit, rather than treating with broad blanket statements. In some instances treating with provocation using an actual substance may be required. It may be that a particular subject has a more complete, and uneventful clearing of allergens by addressing a narrower spectrum of allergens at a particular treatment visit.

2. “Are there active, inactive, or latent PSPs associated with this complaint?” If no, proceed to the next question. If yes, proceed to the following sub-menu. Be aware that PSP issues may present at any fork in a clinical pathway and this is suggested by inappropriate responses to MRT:

a. “Is there any fault in cognitive valuation compromising the perception of the full spectrum of PSPs related to this condition?” If no, proceed to the next question. If yes, proceed to the following sub-menu:

i. “Is any level of the ACS aware of correct cognitive valuation with regard to this?” If yes, proceed to the next question.

ii. “Would it be of benefit to treat with the intention of making all levels of the ACS aware of correct cognitive valuation in this regard?” If yes, treat for this statement, check for correction, and proceed to the next question.

iii. “Would it be of benefit to treat with the intention of correcting all faults in cognitive valuation with regard to this?” If yes, treat for this statement, check for correction, and proceed to the next question.

b. “With regard to the number of active, inactive and latent PSPs is the number 10 to the Xth?” Quantify the PSPs as previously disclosed.

c. “Would it be of benefit at this time to treat with the intention of making all levels of the ACS fully aware of the identity and existence of these PSPs, that they are producing harmful behavior, and that they must be disorganized to restore normal function?” If yes, treat for this statement, recheck for correction, and proceed to the next question.

3. “Are all levels of the ACS fully aware of the identity and existence of, a) all exogenous allergens (inhalant, ingestant, contactant, or injectant substances), or b) all endogenous allergens (any body tissue, body chemical, or breakdown product of the body)?” Alternatively, or additionally specify particular substances of interest (or circumstances, events, places, or times) to see if the ACS is specifically aware of allergy phenomena associated with the named substance. The practitioner may reference exposure to substances at a particular time, or event, with or without naming a specific substance. If yes, proceed to question #4. If no, proceed to the following sub-menu:

a. “Is any level of the ACS fully aware of the existence and identity of all referenced allergens?” If yes, proceed with next question. If no, investigate the reason for this.

b. “Is the immune system fully aware of the existence and identity of all referenced allergens?” If yes, or no, proceed to the next question.

c. “Would it be of benefit to treat with the intention of making all levels of the ACS fully aware of the existence and identity of all referenced allergens, and to communicate this information completely, and immediately to the immune system?” If yes, treat for this command, recheck for correction, and proceed to the next question.

4. “Is there any fault in the tagging of afferent stimuli specific to any, or all referenced allergenic triggers in your exposure history?” Alternatively, this may be asked relative to a particular exposure. If no, proceed to question #6. If yes, proceed to the following sub-menu:

a. “Is this fault afferent path specific?” If not, the fault is global (most commonly). Conceivably, the fault may be specific to a given afferent, eg. chemoreceptor of digestive mucosa. So, proceed to the next question. If yes, proceed to the following sub-menu:

i. MRT query for the appropriate tissue(s) and afferent(s), eg. respiratory mucosa chemoreceptor, and proceed to the following question.

b. “Are all levels of the ACS fully aware of appropriate tagging protocol for these stimuli?” If yes, proceed to the next question. If no, proceed to the following sub-menu:

i. “Is any level of the ACS fully aware of appropriate tagging protocol for these stimuli?” If yes, go to the following question.

ii. “Would it be of benefit at this time to treat with the intention of causing all levels of the ACS to be fully aware of appropriate tagging protocol for all allergen triggers?” If yes, treat spinal points with breathing cycles, confirm correction, and proceed to the next question.

c. “Is inappropriate tagging of afferent stimuli relative to referenced allergenic triggers occurring at (specify brain areas—this is usually amygdala and/or basal ganglia, and sometimes reticular formation, or other levels)?” If yes, proceed to the following sub-menu:

i. “Would it be of benefit at this time to treat with the intention of correcting all inappropriate tagging processes for referenced allergenic triggers occurring at (specified brain site/s)?” Perform treatment, recheck for correction, and proceed to the next question.

5. “Are all levels of the ACS fully aware of the distinction between all referenced allergens and all body tissues?” If yes, proceed to the next question. If no, proceed to the following sub-menu:

a. “Is any level of the autonomic control system fully aware of the distinction between all referenced allergens, and all body tissues?” If no, investigate possible reasons for this. If yes, proceed to next question.

b. “Would it be of benefit to treat at this time with the intention of causing all levels of the ACS to be fully aware of the distinction between all referenced allergens, and all body tissues?” Perform treatment, recheck for correction, and proceed to the next question.

6. “Is there any crossover autoimmune response at this time between any allergen and any body tissue?” If no, proceed to the next question. If yes, proceed to the following sub-menu:

a. “Is this crossover autoimmune reaction caused by a cross linking of data at the (reticular activating system, basal ganglia, and occasionally amygdala)?” If yes, proceed to the next question. If no, check for any other location this may be occurring.

b. “Do all levels of the ACS have sufficient information to correct this cross-linked data?” If yes, proceed to the next question. If no, proceed to the following sub-menu:

i. “Does any level of the ACS have sufficient information to correct this cross-linked data?” If yes, proceed to the next question. If no, investigate any possible correction for this.

ii. “Would it be of benefit at this time to treat with the intent of making all levels of the ACS fully aware of the information necessary to correct the indicated cross linking?” Treat for this statement, recheck, and proceed to next question.

c. “Would it be of benefit to treat at this time with the intention of correcting all cross-linked data in the (specified brain site) causing, or contributing to this crossover autoimmune response between referenced allergens, and all body tissues?” If yes, treat spinal points with breathing cycles, recheck for correction, and proceed to the next question. If no, check for possible reasons such as MRT, PSPs, cog. val., etc.

7. Consider faults in elements of the Sensory/Motor Pathway for the allergy subject. The allergy condition produces recurrent stimulation of sensors in the effected tissues. This results in inappropriate sensor settings, facilitation/inhibition issues that are amenable to SMP intervention. SMP treatment may be necessary to resolve all allergy symptoms, even when the AP has otherwise been cleared.

8. Remember that PSPs may reproduce a real and severe set of allergy symptoms even after the subject has once reached a symptom free status should there be sufficient PSPs that arise to active levels. Recheck the AP if symptoms return assessing any component of the pathway that may prove to be incompletely corrected after the subject is reexposed to their usual environment. Some subjects may experience PSP related symptom return even after the AP is otherwise completely clear.

Provocative Challenges in allergy treatment.

It may be desirable to treat with provocative challenges during the course of treatment. In such cases, treat the allergy pathway in the usual way. Then, in a controlled environment, introduce the substance such as by scent. Go through the protocol again to see if this challenge reveals: awareness of the presence of an allergen, a fault in afferent tagging, inability to distinguish allergen from body tissue, or a crossover response. Retreat as needed to correct any faults indicated and recheck.

The Infectious Agent Pathway (IAP)

It is recognized within the science of microbiology that pathogens of many types, bacteria, molds, and other organisms may undergo pleomorphic changes. These changes cause such organisms to shed their cell wall and adopt a thin cell membrane that is does not have the structure of the parent organism and does not have the signature chemicals that readily identify it to the immune system as a pathogen. Such organisms are often referred to as “stealth pathogens” because they are difficult for the immune system to recognize. Many chronic degenerative diseases such as arthritis, multiple sclerosis, and various digestive system diseases, among others are recognized to be the result of the destructive effect of the body's immune system on tissues in which such stealth pathogens have produced immunologic confusion.

When the immune system is ineffective in dealing with a particular pathogen it is not so much that the immune system does not have powerful enough tools to destroy the structure of the pathogen so much as it is a matter of error in recognition of the pathogen. In the practice of the invention, the infectious agent pathway is designed to assess any faults of the ACS with respect to recognition of infectious agents, to cause the appropriate distribution of this recognition within the ACS such that an appropriate immune system response to the infectious agent may be mounted, and to resolve any confusion of the immune system that may be producing immune system insult to body tissues in the locations where the ACS has the perception such infectious agents may exist.

A best mode outline of treatment for immune system or infections agent dysfunction is described below. The statements or algorithms provided are as listed in Table 4 with directive instruction to facilitate use and understanding of the practice of the invention. It is preferred to begin this pathway with a general check for any fault in cognitive valuation compromising awareness of the presence of infectious agents.

1) “With regard to (state problem), is there an infectious agent causing, or contributing to this condition?” Or, in the clear, “Is any level of the ACS aware of infectious agents (check intracellular lAs separately) resident in (specify organ, tissue system, or other structure). The practitioner must have a clear comprehension of the characteristics and nature of all types of infectious agents to form a clear OTC/OTC interrogatory statement. Be aware of characteristics of types, which include: Bacteria/Mycoplasma/Nanobacteria, Mold, Fungus, Virus/Prion, Amoeba, Parasite. If yes, proceed to the next question. If MRT indicates no, this is not an infectious agent pathway problem, continue with other lines of investigation.

The practitioner should MRT to find all tissue systems harboring infectious agents involved in the condition of interest. There may be pathogens resident in tissue systems other than the tissue system demonstrating the obvious symptoms. It is helpful to MRT to determine the perceptions of the ACS with regard to the types of infectious agents present. This may be of value in suggesting tissue systems harboring the same infectious agents.

The actual determination of any resident infectious agents, and the identification of such pathogens require appropriate laboratory testing. Neuromodulation therapy utilizing the IAP is not a substitute for such medical laboratory testing.

If MRT indicates that all of the infectious agents in a tissue system may not be treated check to see if some categories of infectious agents in the tissue system may be treated.

Several infectious agents may be present in any organ, or tissue system. ACS awareness of other infectious agents may not be apparent until after the most problematic infectious agent has been treated. It is also possible that, because infectious agents inhibit one another, that the immune system may successfully target infectious agents noted when the IAP is first treated, eliminate them, and by so doing enable a previously suppressed set of IAs. If such IAs rise to a sufficient population level at recheck these IAs may now indicated for treatment with the IAP. This does not indicate any failure of previous treatment. Conversely it represents the capacity of the disclosed invention to follow changes in the subject's physiology, and address the dynamics of the disease process. MRT will generally indicate such IAs were present at the time of previous treatment, but were not noted by the ACS.

It is preferred to treat only one tissue system per office visit, and make sure on the subsequent encounter with the subject that MRT indicates clearly that treating IAP for another tissue system is appropriate at that time.

2) “Are there active, inactive, or latent PSPs associated with this complaint?” If no, proceed to the next question. If yes, proceed to the following sub menu, and also be aware that PSP issues may present at any fork in a clinical pathway suggested by inappropriate responses to MRT.

a) “Is there any fault in cognitive valuation compromising the perception of the full spectrum of PSPs related to this condition?” If no, proceed to the next question. If yes, proceed to the following sub-menu:

i) “Is any level of the ACS aware of correct cognitive valuation with regard to this?” If yes, proceed to the next question.

ii) “Would it be of benefit to treat with the intention of making all levels of the ACS aware of correct cognitive valuation in this regard?” If yes, treat for this statement, check for correction, and proceed to the next question.

iii) “Would it be of benefit to treat with the intention of correcting all faults in cognitive valuation with regard to this?” If yes, treat for this statement, check for correction, and proceed to the next question.

b) “With regard to the number of active, inactive and latent PSPs is the number 10 to the Xth?” Quantify PSPs, as disclosed above.

c) “Would it be of benefit at this time to treat with the intention of making all levels of the ACS fully aware of the identity, and existence of these PSPs, that they are producing harmful behavior, and that they must be disorganized to restore normal function?” If yes, treat for this statement, recheck for correction, and proceed to the next question.

3) “Are all levels of the ACS fully aware of the existence, identity, and location of this infectious agent(s)?” If yes, proceed to the next question. If no, proceed to the following sub-menu:

a) “Is any level of the ACS fully aware of the existence, identity, and location of this infectious agent?” If yes, proceed to the next question. If no, isolate the reason and correct for this before proceeding. (Toxicity? PSP? Other?)

b) “Would it be of benefit to treat at this time with the intention of causing all levels of the ACS to be fully aware of the existence, identity, and location of this infectious agent?” If yes, treat for this statement, recheck previous question regarding full awareness, and proceed to the next question once awareness is established. If no, investigate the reason for this.

4) “Would it be of benefit at this time to treat with the intention of instructing the immune system to accurately and completely identify, locate, target, and destroy this infectious agent wherever it exists in the body?” If no, check whether this must be done at another time and when that time is. If yes, perform treatment for this statement, and go to following question.”

5) Is the immune system actively targeting and destroying this infectious agent?” If yes, proceed to the next question. If no, recheck previous steps.

6) “Will any of the infectious agent survive this process of the immune system?” If no, proceed to the question regarding tagging of afferent stimuli. If yes, ask “Have all previous steps of the infectious agent pathway been completed successfully?” If response is still yes, proceed to immediate sub-menu:

a) “Is the infectious agent susceptible to a properly targeted immune system attack?” If no, the subject may require natural, or pharmaceutical antibiotic.

b) “Is there any fault in the function, or regulation of the immune system?” If yes, investigate for autonomic deregulation, toxicity, endocrine influence, depletion, PSPs, or other compromise, and correct.

7) “Is there any fault in the tagging of afferent stimuli related to the perception of the infectious agent?” Be aware that this step may be indicated, but not commonly. If no, proceed to the next question. If yes, proceed to following sub-menu:

a) “Is this fault afferent specific?” If no, the fault is global, so proceed to the following question. If yes, MRT query to find the appropriate tissue, and afferent to facilitate ACS awareness, and proceed to the next question.

b) “Are all levels of the ACS fully aware of appropriate tagging protocol for these stimuli?” If yes, proceed to the next question. If no, proceed to the following sub-menu:

i) “Is any level of the ACS fully aware of appropriate tagging protocol for these stimuli?” If yes, proceed to the next question.

ii) “Would it be of benefit at this time to treat with the intention of causing all levels of the ACS to be fully aware of appropriate tagging protocol for these stimuli?” If yes, treat for this statement, confirm correction, and proceed to the next question.

c) “Are inappropriate tagging processes relative to these stimuli occurring at (specified nervous system site which will usually be amygdala, basal ganglia, or reticular activating system)?” If yes, proceed to the following sub-menu. If no, identify where this is occurring.

i) “Would it be of benefit at this time to treat with the intention of correcting all inappropriate tagging of afferent stimuli associated with the perception of the indicated infectious agent occurring at (specified site)?” If yes, treat for this statement, recheck, and proceed to the next question.

8) “Are all levels of the ACS able to accurately differentiate body tissue from pathogen?” If yes, proceed to the next question. If no, proceed to following sub-menu:

a) “Is any level of the ACS able to accurately differentiate body tissue from pathogen?” If yes, proceed to the following question. If no, investigate the reason for this (MRT, PSPs, cog. val)

b) “Would it be of benefit at this time to treat with the intention of causing all levels of the ACS to be fully aware of the distinction between infectious agent and body tissue, and to communicate this information completely and immediately to the immune system?” If yes, treat for this statement, recheck for correction, and proceed to the next question.”

9) “Is there a crossover autoimmune response between infectious agent and body tissue?” If no, the IAP is completed. Some infectious agents may not involve a crossover autoimmune response—particularly if of recent onset—in which case IAP is primarily directed at training the immune system to target a pathogen it has been unaware of. If yes, proceed to the following sub-menu:

a) “Is this crossover autoimmune reaction caused by a cross linking of data at the (reticular activating system, basal ganglia, and occasionally amygdala)?” If yes, proceed to the next question. If no, check for any other location this may be occurring.

b) “Do all levels of the ACS have sufficient information to correct this cross-linked data?” If yes, proceed to the next question. If no, proceed to the following sub-menu:

i) “Does any level of the ACS have sufficient information to correct this cross-linked data?” If yes, proceed to the next question. If no, investigate any possible correction for this.

ii) “Would it be of benefit at this time to treat with the intent of making all levels of the ACS fully aware of the information necessary to correct the indicated cross linking?” Treat for this statement, recheck, and proceed to next question.

c) “Would it be of benefit to treat at this time with the intention of correcting all cross-linked data in the (specified brain site) causing, or contributing to this crossover autoimmune response between referenced infectious agents, and any body tissues?” If yes, treat spinal points with breathing cycles, recheck for correction, and proceed to the next question. If no, check for possible reasons such as MRT, PSPs, cog. val., etc.

10) “Is there any fault in the sensory/motor pathways associated with the infectious agent phenomena?” If yes, proceed to the sensory/motor pathway protocol. Be aware that the effects of this experience with infectious agent, and the immune system response to the infectious agent may disturb settings of SEOs, and the CNS processing of their afferents. SMP may be required to resolve all symptoms associated with this experience. If no, proceed to the next question.

11) “Is there an autoimmune inflammatory response to any body tissue, body chemical, or breakdown product of the body?” If so, treat the endogenous AP.

Recheck on a subsequent visit to make sure the infectious agent is no longer active in the body, or that the immune system is in the process of locating and destroying the infectious agent, and that none of the infectious agent will survive this activity of the immune system. Also, on reevaluation, check for the existence of any other infectious agents that were not identified, and consequently not the subject of previous IAP treatment. Treat the IAP as needed to clear each tissue system of infectious agents.

Check for the persistence of any fault in the tagging of afferent stimuli associated with the perception of the infectious agent.

Check for the persistence of any portion of the originally noted cross-linked data that may be perpetuating a crossover autoimmune response between body tissues and infectious agent—even after the infectious agent has been eliminated.

Check to see if the infectious agent, or any chemical product of the infectious agent is functioning as an allergen. Follow the Allergy Pathway as indicated.

The Exogenous Analog Pathway (EAP)

Control chemicals exist in the body. These are generally local tissue level hormones, or systemic hormones, usually those from endocrine organs. These chemicals exert their influence by way of the chemical/structural relationship of the control chemical to the chemical functioning as a receptor site on the membrane of the cell whose function is to be controlled.

Chemicals exist in the external environment that have chemical structure sufficiently similar to naturally occurring body control chemicals to mimic their properties. They are capable of coupling with receptor site chemicals on the cells of the body. These chemicals, which may mimic the structure of naturally occurring body control chemicals, will be referred to as “exogenous analogs”

Two Types of Exogenous Analogs:

Inhibitory Exogenous Analogs—These exogenous analogs are close enough to the structure of natural control chemicals to bind to the receptor site; but not close enough in structure to trigger the cellular response of the natural control chemical. The influence of such chemicals in the body is to occupy otherwise available receptor sites, and compromise in an inhibitory way, the expected tissue influence of any given level of naturally occurring hormone/control chemical. The effect occurs by decreasing the probability that an active control chemical will have an opportunity to couple with a tissue receptor site.

Excitatory Exogenous Analogs—These exogenous analogs are close enough to the structure of natural control chemicals to both bind to the receptor site, and to trigger the cellular response of the natural control chemical. The influence of such chemicals in the body is to increase the probability that a tissue receptor site will be triggered. This results in compromise of an excitatory nature of the expected tissue influence of any given level of naturally occurring hormone/control chemical.

Endogenous Analogs—Be aware that the release of endogenous substances in the wrong place, the wrong time, and in the wrong concentrations may result in compromise of normal hormonal control of tissues. This may occur when endogenous substances function as analogs of other control chemicals, and adversely influence body function. In such cases, simply substitute the term “endogenous analog”, or perhaps more specifically MRT for the particular substance (eg. neurotransmitters generally), or specific neurotransmitters, then run the EAP referencing these substances.

The exogenous analog pathway is a clinical algorithm that is structured much like the toxic agent pathway. It is designed to cause of the body to purge and eliminate exogenous analog chemicals in the way that the toxin pathway is described to train the body to eliminate other types of toxins.

A best mode outline of treatment for dysfunction associated with exogenous analog chemicals is described below. The statements or algorithms provided are as listed in Table 5 with directive instruction to facilitate use and understanding of the practice of the invention.

1. “Are all levels of the ACS fully aware of the distinction between naturally occurring control chemicals of the body, and exogenous analogs of such control chemicals?” If yes, proceed to next question. If no, proceed to the following sub-menu:

a. “Is any level of the ACS fully aware of the distinction between naturally occurring, and exogenous analogs of such control chemicals?” If no, investigate the reason for this (Perhaps no such chemicals exist in the body? PSPs?). If yes, proceed to the following question.

b. “Would it be of benefit at this time to treat with the intention of making all levels of the ACS fully aware of the distinction between naturally occurring, and exogenous analogs of such chemicals?” If no, investigate the reason for this (MRT, PSPs, Cog. Val.) If yes, treat for this statement, recheck for correction, and proceed to next question.

2. “Is the ACS at any level aware of the existence of any such exogenous analogs in the body, and the existence, identity, and location of cells in the body coupled to such exogenous analogs?” Alternatively, the question may be made specific to a particular tissue, and/or chemical. If no, pursue another line of investigation. If yes, proceed to the following sub-menu:

a. “Are all levels of the ACS aware of this?” If no, go to the following question. If yes, go to next level question.

b. “Would it be of benefit to treat with the intention of making all levels of the ACS aware of this?” If no, investigate the reason for this. If yes, treat for this statement, recheck for correction, and proceed to the following question.

3. “Is it within the capacity of the ACS to force a purging and release of these exogenous analogs from their binding sites on cells of the body, facilitate their transport away from the tissues, and expedite their degradation and elimination from the body?” If yes, proceed to the next question. If no, investigate the reason for this (MRT, Cognitive Valuation, PSPs?).

4. “Would it be of benefit to treat with the intent of causing the ACS to force the purging of all exogenous analogs from all receptor sites on body tissues and chemicals, to facilitate the transport of these substances from the tissues, and expedite the degradation/elimination of these from the body?” If no, investigate the reason for this (MRT, PSPs, Cognitive Valuation?). If yes, treat, recheck, and proceed to the next question.

5. “Would it be of benefit at this time to treat with the intention of causing the ACS to perpetuate the selective inhibition of re-uptake of any such exogenous analogs from the digestive tract?”

6. “Would it be of benefit at this time to treat with the intention of causing the ACS to perpetuate an awareness for all future presentations of coupled, and uncoupled exogenous analogs and upon recognition purge them from their receptor sites, facilitate their transport away from the tissues, and expedite their elimination/degradation from the body?” If no, investigate the reason for this. If yes, treat this statement.

The Toxin Pathway/Toxin Associated Dysfunction

It is recognized that many substances are toxic to the body. Often this toxicity is based on the capacity of a toxin to bind to a process chemical such as an enzyme and thereby disrupt some physiological process. It is also recognized that the body has the capacity to structure and produce enzymes that have the ability to pull such toxic chemicals away from the sites in the body to which they have bound. The body thereby has inherent to its structure chemical systems to rid the body of poisons. This system may be compromised by any failure to recognize the identity and existence of toxic agents in the internal environment or by a failure to recruit the production of the appropriate enzymes to remove toxins from binding sites in tissues and to transport and eliminate the toxins. The toxic agent pathway assesses whether the ACS accurately perceives the existence of toxins in the body, whether that information is appropriately distributed so that it may result in effective physiological control, and causes the ACS to so distribute the information. This clinical pathway trains the body to chemically process the toxins out of the system. Further, this pathway trains the ACS to selectively block the reuptake of any such subject toxins. Finally, this pathway trains the ACS to maintain awareness for future presentation of the subject toxins, and upon recognition to effect these processes by which toxins may be purged from the body.

The toxin pathway is a tool that can be directed toward any substance that is identified by MRT to be compromising the subject's health. That may include industrial chemicals, endogenous pain producing and potentiating chemicals, heavy metals, industrial chemicals, or just about anything else. MRT with the Toxin Pathway can be as general as a particular class of compounds, or can be used in a more specific manner to check through lists of suspected compounds and substances. The critical issue, as in all NMT investigation, is the understanding of the practitioner of the characteristics of that which is being investigated and the formation of a clear OTC/OTC transmission of that understanding. Such an approach to MRT has the identical value of checking the subject with any number of energetically prepared vials since it is not the energetic properties of the vials that is transmitted to the subject, but the practitioner's comprehension of the subject item.

A best mode outline of treatment for dysfunction associated with toxic agents is described below. The statements or algorithms provided are as listed in Table 6 with directive instruction to facilitate use and understanding of the practice of the invention.

1. “Is the ACS at any level fully aware of the existence, identity, and location in the body of any of the following chemical agents bound to body tissues, or body chemicals and compromising optimal health:”
Exotoxins and/or endotoxins of pathogen origin bound to elements of the nervous system, or other body tissues and chemicals (and functioning as neurotoxins, or other tissue poison);
Immunoglobulins, Immune Complexes (IC's), and Circulating Immune Complexes (CIC's);
Metabolic breakdown products of ingestants (eg. casieomorphines from milk);
Heavy metals;
Halogenated organic compounds (pesticide/herbicide), VOCs (volatile organic compounds);
Pain producing, or potentiating endogenous chemicals;
Other specified toxic agents.

Make note of positive findings regarding categories of toxins present.

2. “Are all levels of the ACS aware of the existence, identity, and location of all such toxic agents?” If yes, proceed to the following question. If no, proceed to the following sub-menu:

a. “Would it be of benefit at this time to treat with the intention of making all levels of the ACS aware of the existence, identity, and location of all such toxic agents?” If no, investigate the reason for this (MRT, Cognitive Valuation, PSPs). If yes, treat for this statement, check for correction, and proceed to the next question.

3. “Is it within the capacity of the ACS to force a purging and release of these toxic agents from their binding sites on body tissues and chemicals, facilitate their transport away from the tissues, and expedite their degradation and elimination from the body?” If yes, proceed to the next question. If no, investigate the reason for this (MRT, Cognitive Valuation, PSPs?).

4. “Should all referenced toxic agents be the subject of treatment at this time?” If yes, proceed to the next question. If no, proceed to the following sub-menu:

a. “Should the following toxic agents be the subject of treatment at this time: (specify toxins found in earlier MRT)?”

5. “Would it be of benefit at this time to treat with the intention of causing the ACS to force a purging and release of these toxic agents (specify appropriate toxins) from their binding sites on body tissues and chemicals, facilitate their transport away from the tissues, and expedite their degradation and elimination from the body; and to do so consistent with the body's capacity to safely tolerate this action?” If no, investigate the reason for this (MRT, Cognitive Valuation, PSPs). If yes, treat for this statement, check for correction, and proceed to the next question.

6. “Would it be of benefit at this time to treat with the intention of causing the ACS to perpetuate the selective inhibition of re-uptake of any such toxic agents from the gastrointestinal tract, urinary tract, or skin?” If no, investigate the reason for this (MRT, Cognitive Valuation, PSPs). If yes, treat for this statement, check for correction, and proceed to the next question.

7. “Would it be of benefit at this time to treat with the intention of causing the ACS to perpetuate an ongoing awareness for future presentation of any such toxic agents and upon recognition to force a purging and release of these toxic agents from their binding sites on body tissues and chemicals, facilitate their transport away from the tissues, and expedite their degradation and elimination from the body; and to do so consistent with the body's capacity to safely tolerate this action?” If yes, treat for this statement, and recheck for correction. If no, investigate the reason for this.

Treating Dysfunctional Morphic Fields

Various levels of control systems exist within the body. The nervous system and the system of hormonal controls distributed by the circulatory system are among these. It has been recognized for thousands of years that energetic systems of control also exist. Traditional Chinese medicine and Ayurvedic medicine were built with an awareness of energetic systems of body control. A relatively new area of science known as formative causation describes the way in which information rich energetic fields known as “morphogenic fields” help to direct the form of structures in the body, even at the molecular level. These morphogenic fields have been used to describe even such processes as protein folding that gives proteins and enzymes there chemical properties.

The inventor discloses that within the scope of the invention that when any morphic field represented entities come together to form a functional relationship that they in turn create their own nested hierarchy of morphic fields (NHMF), a complex morphic field in its own right. The strength of influence of the overarching morphic field on the component morphic field entities relate to the number of elements of similarity between the field components, and the historical duration of those relationships. So, various criteria may influence the degree to which relational morphic field effects are distributed in any system and the strength of their effect.

Formative causation also postulates ancestral morphic fields for all that exists. All things that exist have existed for some time before. Human beings have existed for some few million years in one incarnation, or another; crocodiles and sharks for many millions of years, and the electrons and simpler elements like hydrogen for billions of years.

Under this model human and animal life is a nested hierarchy of morphic fields, a concept that meshes with its analog in the world of neurology. In that world the mind is considered to be a nested hierarchy of neurological structures interacting, functionally resonating between one another, and in so doing bringing into being the characteristics of the larger unit of neurological function.

In this model, by which we exist as a nested hierarchy of energetic fields, is the potential for perturbation of phasic resonance within this nested hierarchy, with the potential for attendant adverse consequences to our health.

Simultaneously, there exists the potential for perturbation between the nested hierarchy of morphic fields, which constitutes ourselves, and the unified morphic field of the rest of the universe. Similarly, there exists the potential for perturbation between the morphic fields of ourselves with any person, place, or thing. All of these are, by definition, complexes of morphic fields. Since, by forming any degree of relationship with such entities, we become part of some higher level morphic field that describes all such components together, we must consider the possibility of pattern perturbation within this complex. There is even the potential for our present nested hierarchy of morphic fields to experience pattern perturbation with our ancestral nested hierarchy of morphic fields, as the effect of such fields spans time, and thus be compromised in the optimal expression of what we are meant to be.

The morphic field pathway is a tool to create energetic harmony within the subject, the subject's ancestral morphic fields, and between the subject and the components of the world within which the subject exists. All that exists is defined in terms of energetic fields. Thus, the morphic field pathway is also a tool to address any disharmony in the relationship of any set of entities. To the degree that such disharmony relates to a pattern perturbation in the morphic resonance between the fields that comprise the subject entities, such disharmony can be turned to harmony.

The NMT morphic field pathway is structured to assess disturbances in the relationship of the various morphic fields of the body and to correct for the “pattern perturbations” or phasic disharmony that adversely influences the function of these fields.

A best mode outline of treatment for morphic field dysfunction is described below. The statements or algorithms provided are as listed in Table 7 with directive instruction to facilitate use and understanding of the practice of the invention.

1. “Is any level of the ACS fully aware of the existence, and identity of the nested hierarchy of morphic fields of the body, and its constituent subsets?” If no, investigate the reasons for this (cognitive valuation, PSPs, MRT). If yes, “Are all levels of the ACS aware of this?” If yes, proceed to the next question. If no, proceed to the following sub-menu:

a. “Would it be of benefit to treat at this time with the intent of making all levels of the ACS fully aware this?” If yes, treat for this statement, recheck and go to the next question.

2. “Is any level of the ACS aware of pattern perturbation in morphic resonance of the nested hierarchy of morphic fields of the body and its constituent subsets?” If no, investigate the reasons for this (cognitive valuation, PSPs, MRT). If yes, “Are all levels of the ACS aware of this?” If yes, proceed to the next question. If no, proceed to the following sub-menu:

a. “Would it be of benefit to treat at this time with the intent of making all levels of the ACS fully aware of this?” If no, investigate the reasons for this (cognitive valuation, PSPs, MRT). If yes, treat for this statement, recheck and go to the next question.

3. “Is the ACS at any level aware of an optimal pattern of resonance for the nested hierarchy of morphic fields of the body (NHMFB), and its constituent subsets?” If no, investigate the reasons for this (cognitive valuation, PSPs, MRT). If yes, “Are all levels of the ACS aware of this?” If yes, proceed to the next question. If no, proceed to the following sub-menu:

a. “Would it be of benefit to treat at this time with the intent of making all levels of the ACS fully aware of this?” If yes, treat for this statement, recheck and go to the next question. If no, investigate the reasons for this (cognitive valuation, PSPs, MRT).

b. “Would it be of benefit to treat at this time with the intent of causing the ACS to synchronize the nested hierarchy of morphic fields of the body, and its constituent subsets to an optimal pattern of morphic resonance?” If no, investigate the reasons for this (cognitive valuation, PSPs, MRT). If yes, treat for this statement, check for correction, and proceed to the next question.

4. “Is any level of the ACS fully aware of the existence, and identity of the unified morphic field, and its constituent subsets?” If yes, “Are all levels of the ACS aware of this?” If yes, proceed to next question. If no, proceed to the following sub-menu:

a. “Would it be of benefit to treat at this time with the intent of making all levels of the ACS fully aware of this?” If yes, treat for this statement, recheck, and proceed to the next question. If no, investigate possible reasons for this (cognitive valuation, PSPs, MRT).

5. “Is any level of the ACS aware of pattern perturbation between the nested hierarchy of morphic fields of the body, and its constituent subsets, and the unified morphic field, and its constituent subsets?” If no, the basic morphic field pathway is concluded. Be aware that it is possible to evaluate and treat with similarly constructed semantic statements for disharmony of the morphic fields of any set of entities, eg. persons, places, things, etc. If yes, “Are all levels of the ACS aware of this?” If yes, proceed to the next question. If no, proceed to the following sub-menu:

a. “Would it be of benefit to treat at this time with the intent of making all levels of the ACS fully aware of this?” If yes, treat for this statement, recheck, and proceed to the next question. If no, investigate possible reasons for this (cognitive valuation, PSPs, MRT).

6. “Is any level of the ACS fully aware of an optimal pattern of resonance between the nested hierarchy of morphic fields of the body, and its constituent subsets, and the unified morphic field, and its constituent subsets?” If no, investigate possible reasons for this (cognitive valuation, PSPs, MRT). If yes, “Are all levels of the ACS aware of this?” If yes, proceed to the next question. If no, proceed to the following sub-menu:

a. “Would it be of benefit to treat with the intention of causing all levels of the ACS to be fully aware of this?” If yes, treat for this statement, recheck, and proceed to the next question. If no, investigate possible reasons for this (cognitive valuation, PSPs, MRT).

7. “Would it be of benefit to treat with the intention of causing the ACS to synchronize the nested hierarchy of morphic fields of the body, and its constituent subsets, and the unified morphic field, and its constituent subsets to an optimal pattern of morphic resonance?” If yes, treat for this statement, recheck, and proceed to the next question. If no, investigate possible reasons for this (cognitive valuation, PSPs, MRT).

The next two questions are “Future Vigilance” statements with the purpose of keeping the ACS alert for any deviation from optimal energetics within morphic fields related to the subject.

8. “Would it be of benefit to treat with the intention of causing the ACS to maintain vigilance for any future pattern perturbation within the nested hierarchy of morphic fields of the body, and its constituent subsets, and upon recognition to synchronize this system to an optimal pattern of morphic resonance?” If yes, treat for this statement, recheck, and proceed to the next question. If no, investigate possible reasons for this (cognitive valuation, PSPs, MRT).

9. “Would it be of benefit to treat with the intent of causing the ACS to maintain vigilance for any future pattern perturbation between the nested hierarchy of morphic fields of the body, and its constituent subsets, and the unified morphic field, and its constituent subsets, and upon recognition to resynchronize these systems to an optimal pattern of morphic resonance?” If yes, treat for this statement, recheck, and proceed to the next question. If no, investigate possible reasons for this (cognitive valuation, PSPs, MRT). Consider any other possible presentations of phasic dis-resonance of the nested hierarchy of morphic fields that constitute the subject with other specific entities the subject regularly interacts with. It is possible to check specifically for energetic disharmony of the subject to all manner of components of the subject's life, to construct specific corrective protocol statements modeled on this pathway, and to treat to produce an optimal energetic relationship for the subject.

10. “Is any level of the ACS aware of pattern perturbation between the nested hierarchy of morphic fields of the body and its constituent subsets, and the ancestral nested hierarchy of morphic fields of the body, and its constituent subsets?” If no, the basic morphic field pathway is concluded. Be aware that it is possible to evaluate and treat with similarly constructed semantic statements for disharmony of the morphic fields of any set of entities, eg. persons, places, things, etc. If yes, “Are all levels or the ACS aware of this?” If yes, proceed to the next question. If no, proceed to the following sub-menu:

a. “Would it be of benefit to treat at this time with the intent of making all levels of the ACS fully aware of this?” If yes, treat for this statement, recheck, and proceed to the next question. If no, investigate possible reasons for this (cognitive valuation, PSPs, MRT).

11. “Is any level of the ACS fully aware of an optimal pattern of resonance between the nested hierarchy of morphic fields of the body, and its constituent subsets, and the ancestral nested hierarchy of morphic fields of the body, and its constituent subsets?” If no, investigate possible reasons for this (cognitive valuation, PSPs, MRT). If yes, “Are all levels of the ACS aware of this?” If yes, proceed to the next question. If no, proceed to the following sub-menu:

a. “Would it be of benefit to treat with the intention of causing all levels of the ACS to be fully aware of this?” If yes, treat for this statement, recheck, and proceed to the next question. If no, investigate possible reasons for this (cognitive valuation, PSPs, MRT).

12. “Would it be of benefit to treat with the intention of causing the ACS to synchronize the nested hierarchy of morphic fields of the body, and its constituent subsets, and the ancestral nested hierarchy of morphic fields of the body, and its constituent subsets to an optimal pattern of morphic resonance?” If yes, treat for this statement, recheck, and proceed to the next question. If no, investigate possible reasons for this (cognitive valuation, PSPs, MRT).

13. “Would it be of benefit to treat with the intent of causing the ACS to maintain vigilance for any future pattern perturbation between the nested hierarchy of morphic fields of the body, and its constituent subsets, and the ancestral nested hierarchy of morphic fields of the body, and its constituent subsets, and upon recognition to resynchronize these systems to an optimal pattern of morphic resonance?” If yes, treat for this statement, recheck, and proceed to the next question. If no, investigate possible reasons for this (cognitive valuation, PSPs, MRT).

14. Regional Field Fault (RFF) is the concept that there can be anatomical regions (even to the level of a specific chemical or receptor), or functional regions eg. the nervous system, or specific structures of the body that are energetically sequestered and isolated from optimal integration within the NHMFB. This should be distinguished from the concept of pattern perturbation, which is akin to the concept of phasic disresonance. With this understanding ask:

a. “(With respect to the stated complaint) Is any level of the ACS aware of the identity, existence, and location of a Regional Field Fault.” “Are all levels?” Treat as necessary for awareness.

b. “Is this RFF specific to a functional system, anatomical region, specific chemical or chemical system?” MRT for specific identification.

c. “Is any level of the ACS aware of an optimal pattern of morphic resonance that resolves the RFF and integrates the RFF to the NHMFB?” Treat as necessary for awareness.

d. “Would it be of benefit to treat with the intent of causing the ACS to synchronize the NHMFB and specified RFF to an optimal pattern of morphic resonance that integrates the RFF within the NHMFB?” Treat and recheck

When the issue is that the subject is in disharmony with another person, eg. a spouse, friend, child, etc. perform the morphic field pathway for two entities as described above for “Ancestral Morphic Field”, and for “Unified Morphic Field” substituting the name of the person for the “Ancestral” or “Unified” morphic fields.

In such a case it is preferable that each person be synchronized for the basic morphic field pathway first, and then that the two people be synchronized to each other.

When the issue is disharmony between a person and some place, or thing, first synchronize the person with the basic morphic field pathway, and then to that secondary entity.

Then check that all levels of the autonomic control system are aware of the morphic field of the entity to which the individual is to be synchronized. Then synchronize the person to the morphic field of the entity in question using the above examples as a language template for the pathway.

When the issue is disharmony within some group you may attempt to address the collective morphic field of the group using a person as a surrogate to investigate the status of the morphic field of the group as a distinct entity of its own, and synchronize the group, as you would an individual using the above examples as a language template for the pathway.

If it is accepted that there is the concept of the zero-point field, and all that goes with that perspective, many possibilities open up to us therapeutically. This model of the universe is denoted by properties of non-locality. Similarly, this model is characterized by the properties of morphic fields, and the concept of formative causation that holds that ancestral morphic fields representing all that has gone before our current temporal window continues to be energetically connected to the present. Implications of this suggest that all sorts of interesting, if not uncanny, treatment possibilities exist.

It may be of interest to subjects with awareness, and concern in matters of a spiritual nature to synchronize the nested hierarchy of morphic fields of the body, and its constituent subsets with the morphic field of the manifestations of God, eg. Moses, Christ, Buddah, Mohammed, Zoroaster, Krishna, Baha'u'llah separately, or collectively.

While there has been shown and described what is presently considered to be the preferred methods of practicing this invention, it will be obvious to those skilled in the art that various changes and modifications may be made without departing from the broader aspects of this invention. It is, therefore, aimed in the appended claims to cover all such changes and modifications as fall within the true spirit and scope of this invention.

Patent Titled: A Method of Neuromodulation Therapy

Inventor: LESLIE S FEINBERG

TABLE 1
Pernicious Synaptic Patterns (For Any Pathway)

| | |
|---|---|
| 1. | “Are there active, inactive, or latent PSPs associated with this complaint?” Use |
| | semantic cues, position, posture, muscle challenge, and other methods taught to provoke |
| | PSPs to a level of awareness to facilitate treatment. |
| a. | “Is there any fault in cognitive valuation compromising the perception of the |
| | full spectrum of PSPs related to this condition?” |
| i. | “Is any level of the ACS aware of correct cognitive valuation with regard |
| | to this?” |
| ii. | “Are all levels of the ACS aware of correct cognitive valuation with |
| | regard to this?” |
| iii. | “Would it be of benefit to treat with the intention of making all levels of |
| | the ACS aware of correct cognitive valuation in this regard?” |
| iv. | “Would it be of benefit to treat with the intention of correcting all faults |
| | in cognitive valuation at this time?” |
| b. | “With regard to the number of active, inactive, and latent PSPs is the number |
| | 10 to the Xth?” |
| c. | “Would it be of benefit at this time to treat with the intention of making all |
| | levels of the ACS fully aware of the identity and existence of these PSPs, that |
| | they are producing harmful behavior, and that they must be disorganized to |
| | restore normal function?” |
| d. | Perform serial rechecks within a visit session using the methods you know for |
| | provoking them to awareness until no further PSPs are evoked, or satisfactory |
| | symptom improvement is achieved. |
| e. | Future Vigilance Statement may be used: “Would it be of benefit to treat with |
| | the intention of causing the ACS to maintain an awareness for any future |
| | presentation of pernicious synaptic patterns (related to this condition), and |
| | upon recognition to cause the ACS to recognize the identity and existence of |
| | these pernicious synaptic patterns, the fact they are causing harmful behavior, |
| | and that they must be disorganized to permit a return of normal function?” |
| 2. | Provide corrective instruction. |
| a. | Follow with physical stimuli. |

Patent Titled: A Method of Neuromodulation Therapy

Inventor: LESLIE S FEINBERG

TABLE 2
The Sensory/Motor Pathway (SMP)

| | |
|---|---|
| 1. | “With regard to the (specify the sensory/motor complaint, dysfunction, tissue, |
| | organ, or system) is there any fault in sensory/motor control causing, or contributing to |
| | this condition (dysfunction of tissue, or system, etc.)?” |
| a. | “Is there any role of an infectious agent/allergic phenomena (endogenous, or |
| | exogenous allergen)/crossover autoimmune phenomena between infectious |
| | agent and body tissue/or an endogenous/exogenous allergen and body tissue |
| | in (specify problem)?” Tx per indicated pathway. |
| 2. | “Is there any fault in cognitive valuation with regard to any aspect of the |
| | condition?” |
| a. | “Are all levels of the ACS fully aware of correct cognitive valuation of all |
| | aspects of this condition?” |
| i. | “Is any level of the ACS fully aware of correct cognitive valuation of all |
| | aspects of this condition?” |
| ii. | “Would it be of benefit to treat at this time with the intention of making |
| | all levels of the ACS fully aware of the correct cognitive valuation of all |
| | aspects of this condition?” |
| b. | “Would it be of benefit to treat at this time with the intention of correcting all |
| | faults in cognitive valuation of the condition?” |
| 3. | “Are there active, inactive, or latent PSPs associated with this complaint?” |
| a. | “Is there any fault in cognitive valuation compromising the perception of the |
| | full spectrum of PSPs related to this condition?” |
| i. | “Is any level of the ACS aware of correct cognitive valuation with regard |
| | to this?” |
| ii. | “Would it be of benefit to treat with the intention of making all levels of |
| | the ACS aware of correct cognitive valuation/correcting all faults in |
| | cognitive valuation with regard to this?” (as separate, or combined |
| | statement) |
| b. | “With regard to the number of active, inactive and latent PSPs is the number |
| | 10 to the Xth?” |
| c. | “Would it be of benefit at this time to treat with the intention of making all |
| | levels of the ACS fully aware of the identity and existence of these PSPs, that |
| | they are producing harmful behavior, and that they must be disorganized to |
| | restore normal function?” |
| 4. | “Is there any fault in the tagging of afferent stimuli relating to the perception of |
| | this condition?” |
| a. | “Is this afferent specific/global?” |
| b. | “Are all levels of the autonomic control system fully aware of appropriate |
| | tagging protocol for the indicated stimuli?” |
| i. | “Is any level of the ACS fully aware of appropriate tagging protocol for |
| | the indicated stimuli?” |
| ii. | “Would it be of benefit at this time to treat with the intention of causing |
| | all levels of the ACS to be fully aware of appropriate tagging protocol for |
| | these stimuli?” |
| c. | “Are inappropriate tagging processes relative to the indicated stimuli |
| | occurring at (check for the involved nervous system site which will usually |
| | be amygdala, or basal ganglia - possibly RAS)?” |
| i. | “Would it be of benefit at this time to treat with the intention of |
| | correcting all inappropriate tagging of afferent stimuli occurring at |
| | (specified site)?” |
| 5. | “Is there a fault in any sensory end organ (SEO) contributing to this condition?” |
| | eg. “Is there a fault in an SEO of fascia?” “Is this a stretch receptor/pain receptor?” “Is |
| | this receptor set at an inappropriately high/low threshold of sensitivity?” |
| a. | “With regard to (the indicated SEO) is its afferent being inappropriately |
| | facilitated, or inhibited?” Determine the site of this action - generally T, HT, |
| | HC |
| b. | “Can you register this fault?” Continue until all involved SEOs have been |
| | discovered if stacking SEOs. |
| c. | “Would it be of benefit to treat at this time with the intention of resetting |
| | (specify SEOs, or “the indicated SEO/s”) to a higher/lower threshold of |
| | stimulation, and to stop all inappropriate facilitation/inhibition at the (specify |
| | CNS site, or “indicated CNS processing sites?”) |
| d. | Alternatively, if stacking SEOs: “Would it be of benefit at this time to treat |
| | with the intention of resetting all indicated sensory end organs to optimal |
| | sensitivity levels, and to stop all inappropriate facilitiation and inhibition at |
| | the indicated CNS processing areas.” |
| 6. | “Is there any fault in central nervous system processing causing, or contributing |
| | to this condition?” |
| 7. | “Is there any fault in sympathetic, or parasympathetic processing causing, or |
| | contributing to this condition?” Identify dysfunction and construct corrective statement, |
| | eg. “Would it be of benefit to treat at this time with the intention of stopping all |
| | inappropriate facilitation of sacral parasympathetic ganglia at the hypothalamic (HT) |
| | level.” Or, “Would it be of benefit to stop all facilitation of sympathetic efferents from |
| | the stellate ganglia (or, a more general statement referencing any involved sympathetic |
| | ganglia) to muscle wall of cerebral vasculature structures consistent with optimal |
| | function?” |
| 8. | “Is there any fault in motor processing causing, or contributing to this |
| | condition?” Assess CNS facil/inhib and treat, eg. “Would it be of benefit to treat at this |
| | time with the intention of stopping all inhibition of motor efferents to antagonist muscles |
| | in the area of complaint at the thalamus level?” |
| 9. | “Is there any fault in a motor end organ causing, or contributing to this |
| | condition?” Assess high/low threshold of stimulation and treat, eg. “Would it be of |
| | benefit to treat at this time with the intention of resetting the indicated motor end organ of |
| | dermal sweat gland to a high threshold of stimulation consistent with optimal function?” |
| 10. | “Is there any other fault in autonomic regulation causing, or contributing to |
| | this condition?” |
| 11. | Provide corrective instruction. |
| a. | Follow with physical stimuli. |

Patent Titled: A Method of Neuromodulation Therapy

Inventor: LESLIE S FEINBERG

TABLE 3
The Allergy Pathway (AP)

| | |
|---|---|
| 1. | “With regard to the referenced symptoms/condition are there (Or, in the clear, |
| | “Is any level of the ACS aware of . . . ”) any inhalant, ingestant, contactant, or injectant |
| | substances (exogenous allergens) in your exposure history which are capable of behaving |
| | as allergenic triggers? (“ . . . causing or contributing to this condition?”) Always check, |
| | “With regard to the referenced symptoms/condition are there (Or, in the clear, “Is any |
| | level of the ACS aware of . . . ”) any body tissues, body chemicals, or breakdown products |
| | of the body (endogenous allergens) which are capable of behaving as allergenic triggers |
| | (“...causing or contributing to this condition?”) |
| 2. | “Are there active, inactive, or latent PSPs associated with this complaint?” |
| | Perform appropriate correction. |
| 3. | “Are all levels of the ACS fully aware of the identity and existence of a.) all |
| | exogenous allergens (inhalant, ingestant, contactant, or injectant substances), or b.) all |
| | endogenous allergens (any body tissue, body chemical, or breakdown product of the |
| | body)?” |
| a. | “Is any level of the ACS fully aware of the existence and identity of all |
| | referenced allergens?” |
| b. | “Is the immune system fully aware of the existence and identity of all |
| | referenced allergens?” |
| c. | “Would it be of benefit to treat with the intention of making all levels of the |
| | ACS fully aware of the existence and identity of all referenced allergens, and |
| | to communicate this information completely, and immediately to the immune |
| | system?” |
| 4. | “Is there any fault in the tagging of afferent stimuli specific to any, or all |
| | referenced allergenic triggers in your exposure history?” |
| a. | “Is this fault afferent path specific?” If so, find tissue(s) and afferent(s) If |
| | not, the fault is global (most commonly). |
| b. | “Are all levels of the ACS fully aware of appropriate tagging protocol for these |
| | stimuli |
| i. | “Is any level of the ACS fully aware of appropriate tagging protocol for |
| | these stimuli?” |
| ii. | “Would it be of benefit at this time to treat with the intention of causing |
| | all levels of the ACS to be fully aware of appropriate tagging protocol for |
| | all allergen triggers |
| c. | “Is inappropriate tagging of afferent stimuli relative to referenced allergenic |
| | triggers occurring at (specify brain areas-this is usually amygdala and/or basal |
| | ganglia, and sometimes reticular formation, or other levels)?” |
| i. | “Would it be of benefit at this time to treat with the intention of correcting |
| | all inappropriate tagging processes for referenced allergenic triggers |
| | occurring at (specified brain site/s)?” |
| 5. | “Are all levels of the ACS fully aware of the distinction between all referenced |
| | allergens and all body tissues?” |
| a. | “Is any level of the autonomic control system fully aware of the distinction |
| | between all referenced allergens, and all body tissues?” |
| b. | “Would it be of benefit to treat at this time with the intention of causing all |
| | levels of the ACS to be fully aware of the distinction between all referenced |
| | allergens, and all body tissues?” |
| 6. | “Is there any crossover autoimmune response at this time between any allergen |
| | and any body tissue?” |
| a. | “Is this crossover autoimmune reaction caused by a cross linking of data at the |
| | (reticular activating, system basal ganglia, and occasionally amygdala)?” |
| b. | “Do all levels of the ACS have sufficient information to correct this cross- |
| | linked data?” |
| i. | “Does any level of the ACS have sufficient information to correct this |
| | cross-linked data?” |
| ii. | “Would it be of benefit at this time to treat with the intent of making all |
| | levels of the ACS fully aware of the information necessary to correct the |
| | indicated cross linking?” |
| c. | “Would it be of benefit to treat at this time with the intention of correcting all |
| | cross-linked data in the (specified brain site) causing, or contributing to this |
| | crossover autoimmune response between referenced allergens, and all body |
| | tissues?” |
| 7. | Consider faults in elements of the Sensory/Motor Pathway for the allergy |
| | patient. eg., muscosal cheomoreceptors |
| 8. | Remember that PSPs may reproduce as real and severe a set of allergy |
| | symptoms even after the patient has once reached a symptom free status should there be |
| | sufficient PSPs that arise to active levels. Recheck the AP if symptoms return assessing |
| | any component of the pathway that may prove to be incompletely corrected after the |
| | patient is re-exposed to their usual environment. Some patients may experience PSP |
| | related symptom return even after the AP is otherwise completely clear. |
| 9. | Provide corrective instruction. |
| a. | Follow with physical stimuli. |

Patent Titled: A Method of Neuromodulation Therapy

Inventor: LESLIE S FEINBERG

TABLE 4
The Infectious Agent Pathway (IAP)

| | |
|---|---|
| | Always Begin This Pathway With A General Check For Any Fault In Cognitive |
| | Valuation Compromising Awareness Of The Presence Of Infectious Agents. |
| 1. | “With regard to (state problem) is there an infectious agent causing, or |
| | contributing to this condition?” Or, in the clear, “Is any level of the ACS aware of |
| | infectious agents (check intracellular IAs separately) resident in (specify organ, tissue |
| | system, or other structure). Be aware of characteristics of types: |
| | Bacteria/Mycoplasma/Nanobacteria, Mold, Fungus, Virus/Prion, Amoeba, Parasite. |
| 2. | “Are there active, inactive, or latent PSPs associated with this complaint?” |
| | Perform the appropriate correction. |
| 3. | “Are all levels of the ACS fully aware of the existence, identity, and location of |
| | this infectious agent?” |
| a. | “Is any level of the ACS fully aware of the existence, identity, and location of |
| | this infectious agent?” |
| b. | “Would it be of benefit to treat at this time with the intention of causing all |
| | levels of the ACS to be fully aware of the existence, identity, and location of |
| | this infectious agent?” |
| 4. | “Would it be of benefit at this time to treat with the intention of instructing the |
| | immune system to accurately and completely identify, locate, target, and destroy this |
| | infectious agent wherever it exists in the body?” |
| 5. | “Is the immune system actively targeting and destroying this infectious agent?” |
| 6. | “Will any of the infectious agent survive this process of the immune system?” |
| | If no, proceed to the question regarding tagging of afferent stimuli. If yes, ask “Have all |
| | previous steps of the infectious agent pathway been completed successfully?” |
| a. | “Is the infectious agent susceptible to a properly targeted immune system |
| | attack?” |
| b. | “Is there any fault in the function, or regulation of the immune system?” |
| 7. | “Is there any fault in the tagging of afferent stimuli related to the perception of |
| | the infectious agent?” |
| a. | “Is this fault afferent specific?” |
| b. | “Are all levels of the ACS fully aware of appropriate tagging protocol for |
| | these stimuli?” |
| i. | “Is any level of the ACS fully aware of appropriate tagging protocol for |
| | these stimuli?” |
| ii. | “Would it be of benefit at this time to treat with the intention of causing all |
| | levels of the ACS to be fully aware of appropriate tagging protocol for |
| | these stimuli?” |
| c. | “Are inappropriate tagging processes relative to these stimuli occurring at |
| | (specified nervous system site which will usually be amygdala, basal ganglia, |
| | or reticular activating system)?” |
| i. | “Would it be of benefit at this time to treat with the intention of correcting |
| | all inappropriate tagging of afferent stimuli associated with the perception |
| | of the indicated infectious agent occurring at (specified site)?” |
| 8. | “Are all levels of the ACS able to accurately differentiate body tissue from |
| | pathogen?” |
| a. | “Is any level of the ACS able to accurately differentiate body tissue from |
| | pathogen?” |
| b. | “Would it be of benefit at this time to treat with the intention of causing all |
| | levels of the ACS to be fully aware of the distinction between infectious agent |
| | and body tissue, and to communicate this information completely and |
| | immediately to the immune system?” |
| 9. | “Is there a crossover autoimmune response between infectious agent and body |
| | tissue?” |
| a. | “Is this crossover autoimmune reaction caused by a cross linking of data at the |
| | (reticular activating system, basal ganglia, and occasionally amygdala)?” |
| b. | “Do all levels of the ACS have sufficient information to correct this cross- |
| | linked data?” |
| i. | “Does any level of the ACS have sufficient information to correct this |
| | cross-linked data?” |
| ii. | “Would it be of benefit at this time to treat with the intent of making all |
| | levels of the ACS fully aware of the information necessary to correct the |
| | indicated cross linking?” |
| c. | “Would it be of benefit to treat at this time with the intention of correcting all |
| | cross-linked data in the (specified brain site) causing, or contributing to this |
| | crossover autoimmune response between referenced infectious agents, and |
| | any body tissues?” |
| 10. | “Is there any fault in the sensory/motor pathways associated with the |
| | infectious agent phenomena?” |
| 11. | “Is there an autoimmune inflammatory response to any body tissue, body |
| | chemical, or breakdown product of the body?” If so, you will need to treat the |
| | endogenous AP. Check TP as most IAs produce exo/endo toxins. |
| 12. | Provide corrective instruction. |
| a. | Follow with physical stimuli |

Patent Titled: A Method of Neuromodulation Therapy

Inventor: LESLIE S FEINBERG

TABLE 5
NMT: The Exogenous Analog Pathway (EAP)

| | |
|---|---|
| 1. | “Are all levels of the ACS fully aware of the distinction between naturally |
| | occurring control chemicals of the body, and exogenous analogs of such control |
| | chemicals?” |
| a. | “Is any level of the ACS fully aware of the distinction between naturally |
| | occurring control chemicals of the body, and exogenous analogs of such |
| | control chemicals?” |
| b. | “Would it be of benefit at this time to treat with the intention of making all |
| | levels of the ACS fully aware of the distinction between naturally occurring |
| | control chemicals of the body, and exogenous analogs of such chemicals?” |
| 2. | “Is the ACS at any level aware of the existence of exogenous analogs in the |
| | body, and the existence, identity, and location of cells in the body with control chemical |
| | receptor sites coupled to exogenous analogs?” |
| a. | “Are all levels of the ACS aware of this?” |
| b. | “Would it be of benefit to treat with the intention of making all levels of the |
| | ACS aware of this?” |
| 3. | “Is it within the capacity of the ACS to force a purging and release of these |
| | toxic agents from their binding sites on body tissues and body chemicals, facilitate their |
| | transport away from the tissues, and expedite their degradation and elimination from the |
| | body?” |
| 4. | “Would it be of benefit to treat with the intent of causing the ACS to force the |
| | purging of all exogenous analogs from all receptor sites on body tissues and chemicals, to |
| | facilitate the transport of these substances from the tissues, and expedite the degradation/ |
| | elimination of these from the body?” |
| 5. | “Would it be of benefit at this time to treat with the intention of causing the |
| | ACS to perpetuate a selective inhibition of the re-uptake of any such exogenous analogs |
| | from the digestive tract?” |
| 6. | “Would it be of benefit at this time to treat with the intention of causing the |
| | ACS to perpetuate an awareness for all future presentations of coupled, and uncoupled |
| | exogenous analogs and upon recognition to force the purging of all exogenous analogs |
| | from all receptor sites on body tissues and chemicals, to facilitate the transport of these |
| | substances from the tissues, and expedite the degradation/elimination of these from the |
| | body?” |
| 7. | Provide corrective instruction. |
| a. | Follow with physical stimuli. |

Patent Titled: A Method of Neuromodulation Therapy

Inventor: LESLIE S FEINBERG

TABLE 6
The Toxin Pathway

| | |
|---|---|
| 1. | “Is the ACS at any level fully aware of the existence, identity, and location in |
| | the body of any of the following chemical agents bound to body tissues, or body |
| | chemicals and compromising optimal health”: |
| a. | Exo/endo toxins of pathogen origin bound to elements of the nervous system, |
| | or other body tissues and chemicals (and functioning as neurotoxins, or other |
| | tissue poison). |
| b. | Immunoglobulins/Immune Complexes (IC's)/Circulating Immune Complexes |
| | (CIC's) |
| c. | Metabolic breakdown products of ingestants (eg.casieomorphines from milk) |
| d. | Heavy metals |
| e. | Halogenated organic compounds (pesticide/herbicide), VOCs (volatile organic |
| | compounds) |
| f. | Pain producing, or potentiating endogenous chemicals |
| g. | Other specified toxic agents |
| | Make note of positive findings regarding categories of toxins present. |
| 2. | “Are all levels of the ACS aware of the existence, identity, and location of all |
| | such toxic agents (specify categories, or specific toxins)?” |
| a. | “Would it be of benefit at this time to treat with the intention of making all |
| | levels of the ACS aware of the existence, identity, and location of all such |
| | toxic agents?” |
| 3. | “Is it within the capacity of the ACS to force a purging and release of these |
| | toxic agents from their binding sites on body tissues and body chemicals, facilitate their |
| | transport away from the tissues, and expedite their degradation and elimination from the |
| | body?” |
| 4. | “Should all referenced toxic agents be the subject of treatment at this time?” If |
| | yes, proceed to the next question. If no, proceed to the following sub-menu: |
| a. | “Should the following toxic agents be the subject of treatment at this time: |
| | (specify toxins found in earlier MRT)?” |
| 5. | “Would it be of benefit at this time to treat with the intention of causing the |
| | ACS to force a purging and release of these toxic agents (specify appropriate toxins) from |
| | their binding sites on body tissues and chemicals, facilitate their transport away from the |
| | tissues, and expedite their degradation and elimination from the body; and to do so |
| | consistent with the body's capacity to safely tolerate this action?” |
| 6. | “Would it be of benefit at this time to treat with the intention of causing the |
| | ACS to perpetuate a selective inhibition of the re-uptake of any such toxic agents from |
| | the gastrointestinal tract, urinary tract, and skin?” |
| 7. | “Would it be of benefit at this time to treat with the intention of causing the |
| | ACS to perpetuate an ongoing awareness for any future presentation of such toxic agents |
| | and upon recognition to force a purging and release of these toxic agents from their |
| | binding sites on body tissues and chemicals, facilitate their transport away from the |
| | tissues, and expedite their degradation and elimination from the body; and to do so |
| | consistent with the body's capacity to safely tolerate this action?” |
| 8. | Provide corrective instruction. |
| a. | Follow with physical stimuli. |

Patent Titled: A Method of Neuromodulation Therapy

Inventor: LESLIE S FEINBERG

TABLE 7
The Morphic Fields Pathway

| | |
|---|---|
| 1. | “Is any level of the ACS fully aware of the existence, and identity of the nested |
| | hierarchy of morphic fields of the body, and its constituent subsets?” “Are all levels of |
| | the ACS aware of this?” |
| a) | “Would it be of benefit to treat at this time with the intent of making all levels |
| | of the ACS fully aware this?” |
| 2. | “Is any level of the ACS aware of pattern perturbation in morphic resonance of |
| | the nested hierarchy of morphic fields of the body and its constituent subsets?” “Are all |
| | levels of the ACS aware of this?” |
| a) | “Would it be of benefit to treat at this time with the intent of making all levels |
| | of the ACS fully aware of this?” |
| 3. | “Is the ACS at any level aware of an optimal pattern of resonance for the nested |
| | hierarchy of morphic fields of the body, and its constituent subsets?” “Are all levels of |
| | the ACS aware of this?” |
| a) | “Would it be of benefit to treat at this time with the intent of making all levels |
| | of the ACS fully aware of this?” |
| 4. | “Would it be of benefit to treat at this time with the intent of causing the ACS |
| | to synchronize the nested hierarchy of morphic fields of the body, and its constituent |
| | subsets to an optimal pattern of resonance?” |
| 5. | “Is any level of the ACS fully aware of the existence, and identity of the unified |
| | morphic field, and its constituent subsets?” “Are all levels of the ACS aware of this?” |
| a) | “Would it be of benefit to treat at this time with the intent of making all levels |
| | of the ACS fully aware of this?” |
| 6. | “Is any level of the ACS aware of pattern perturbation between the nested |
| | hierarchy of morphic fields of the body, and its constituent subsets, and the unified |
| | morphic field, and its constituent subsets?” “Are all levels of the ACS aware of this?” |
| a) | “Would it be of benefit to treat at this time with the intent of making all levels |
| | of the ACS fully aware of this?” |
| 7. | “Is any level of the ACS fully aware of an optimal pattern of resonance |
| | between the nested hierarchy of morphic fields of the body, and its constituent subsets, |
| | and the unified morphic field, and its constituent subsets?” “Are all levels of the ACS |
| | aware of this?” |
| a) | “Would it be of benefit to treat with the intention of causing all levels of the |
| | ACS to be fully aware of this?” |
| 8. | “Would it be of benefit to treat with the intention of causing the ACS to |
| | synchronize the nested hierarchy of morphic fields of the body, and its constituent |
| | subsets, and the unified morphic field, and its constituent subsets to an optimal pattern of |
| | resonance?” |
| 9. | “Would it be of benefit to treat with the intention of causing the ACS to |
| | maintain vigilance for any future pattern perturbation within the nested hierarchy of |
| | morphic fields of the body, and its constituent subsets, and upon recognition to |
| | synchronize this system to an optimal pattern of morphic resonance?” If yes, treat for |
| | this statement, recheck, and proceed to the next question. If no, investigate possible |
| | reasons for this (cognitive valuation, PSPs, MRT). |
| 10. | “Would it be of benefit to treat with the intent of causing the ACS to maintain |
| | vigilance for any future presentation of pattern perturbation between the nested hierarchy |
| | of morphic fields of the body, and its constituent subsets, and the unified morphic field, |
| | and its constituent subsets, and upon recognition to resynchronize these systems to an |
| | optimal pattern of morphic resonance?” |
| | Alternatively for 9 & 10, “Would it be of benefit to treat with the intention of |
| | causing the ACS to maintain vigilance for any future pattern perturbation within, and |
| | between the nested hierarchy of morphic fields of the body, and its constituent subsets, |
| | and the unified morphic field, and its constituent subsets, and upon recognition to |
| | resynchronize these systems to an optimal pattern of morphic resonance within and |
| | between themselves?” |
| 11. | “Is any level of the ACS aware of pattern perturbation between the nested |
| | hierarchy of morphic fields of the body and its constituent subsets, and the ancestral |
| | nested hierarchy of morphic fields of the body, and its constituent subsets?” “Are all |
| | levels or the ACS aware of this?” |
| a) | “Would it be of benefit to treat at this time with the intent of making all levels |
| | of the ACS fully aware of this?” |
| 12. | “Is any level of the ACS fully aware of an optimal pattern of resonance |
| | between the nested hierarchy of morphic fields of the body, and its constituent subsets, |
| | and the ancestral nested hierarchy of morphic fields of the body, and its constituent |
| | subsets?” “Are all levels of the ACS aware of this?” |
| a) | “Would it be of benefit to treat with the intention of causing all levels of the |
| | ACS to be fully aware of this?” |
| 13. | “Would it be of benefit to treat with the intention of causing the ACS to |
| | synchronize the nested hierarchy of morphic fields of the body, and its constituent |
| | subsets, and the ancestral nested hierarchy of morphic fields of the body, and its |
| | constituent subsets to an optimal pattern of resonance?” |
| 14. | “Would it be of benefit to treat with the intent of causing the ACS to maintain |
| | vigilance for any future presentation of pattern perturbation between the nested hierarchy |
| | of morphic fields of the body, and its constituent subsets, and the ancestral nested |
| | hierarchy of morphic fields of the body, and its constituent subsets, and upon recognition |
| | to resynchronize these systems to an optimal pattern of morphic resonance?” |
| 15. | Provide corrective instruction. |
| a. | Follow with physical stimuli. |
| 16. | Regional Field Fault (RFF) is the concept that there can be anatomical regions |
| | (even to the level of a specific chemical or receptor), or functional regions, or specific |
| | structures of the body that are energetically sequestered and isolated from optimal |
| | integration within the NHMFB. With this understanding ask: |
| a) | “(With respect to the stated complaint) Is any level of the ACS aware of the |
| | identity, existence, and location of a Regional Field Fault.” “Are all levels?” |
| | Tx as necessary for awareness. |
| b) | “Is this RFF specific to a functional system, anatomical region, specific |
| | chemical or chemical system?” MRT for specific ID. |
| c) | “Is any level of the ACS aware of an optimal pattern of morphic resonance |
| | that resolves the RFF and integrates the RFF to the NHMFB?” Tx as |
| | necessary for awareness. |
| d) | “Would it be of benefit to treat with the intent of causing the ACS to |
| | synchronize the NHMFB and specified RFF to an optimal pattern of morphic |
| | resonance that integrates the RFF within the NHMFB?” Tx. and recheck |
| e) | Provide corrective instruction. |
| i. | Follow with physical stimuli. |

1. A method of providing energetic based health care comprising the steps of:
a) providing a subject to be treated with a one or more dysfunction,
b) providing a practitioner,
c) said practitioner forming a pathway statement,
d) said practitioner interrogating said subject to be treated,
e) said practitioner assessing said one or more dysfunction,
f) correcting said one or more dysfunction.

2. The method of claim 1 wherein the interrogating of said subject is accomplished in part through muscle response testing.

3. The method of claim 1 wherein the forming a pathway statement comprises the steps of utilizing one or more semantic algorithms to facilitate the formation of a pathway statement in said practitioner.

4. The method of claim 1 wherein the interrogating of said subject takes place on an other than conscious to other that conscious level.

5. The method of claim 1 wherein the correcting of said one or more dysfunction comprises the steps of providing a corrective command statement.

6. The method of claim 5 wherein the providing of said corrective command statement comprises providing a verbal corrective command.

7. The method of claim 5 wherein the providing of said corrective command statement comprises providing a non-verbal corrective command.

8. The method of claim 1 wherein correcting said one or more dysfunction comprises the additional step providing physical stimuli.

9. The method of claim 1 wherein said subject and said practitioner are the same person.
